SEQUENCE LISTING (6 sequences)

SEQ ID NO: 1 - DNA, 5498 bp, synthetic construct
  misc_feature 1..5498: note = Expression cassette with IBDV-VP2 and NDV-F genes
  source 1..5498: mol_type = other DNA
aactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtagccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacaattatttcatttaataacatatagcccaaagacctctatgaacatttagtttcccgtatactcaacggcgcgtgtacacacaagggcgaattccacagtggatatcaagcttaattaagtaccgagctcgaattggcgcgccaggtcaattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgcgccggatccatgggccccagaccttctaccaagaacccagtacctatgatgctgactgtccgagtcgcgctggtactgagttgcatctgtccggcaaactccattgatggcaggcctcttgcggctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcccgattctagcatgctacctaatgtacaagcaaaaggcgcaacaaaagaccttattatggcttgggaataatactctagatcagatgagagccactacaaaaatgtgaggatctctcgaggaattctagatcccacgtcactattgtatactctatattatactctatgttatactctgtaatcctactcaataaacgtgtcacgcctgtgaaaccgtactaagtctcccgtgtcttcttatcaccatcaggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctcccc

SEQ ID NO: 2 - DNA, 3157 bp, synthetic construct
  misc_feature 1..3157: note = Expression cassette with ILTV gD and gI genes
  source 1..3157: mol_type = other DNA
aagcttaattaacgtcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtgaagatctcttaattaacaataaacatagcatacgttatgacatggtctaccgcgtcttatatggggacgacaagctt

SEQ ID NO: 3 - DNA, 20 bp, synthetic construct
  misc_feature 1..20: note = guide RNA for insertion between UL44 and UL45 genes of HVT
  source 1..20: mol_type = other DNA
acatcgggacgtacatcatg

SEQ ID NO: 4 - DNA, 20 bp, synthetic construct
  misc_feature 1..20: note = guide RNA for insertion between UL45 and UL46 genes of HVT
  source 1..20: mol_type = other DNA
ctaacggttactgtgtttta

SEQ ID NO: 5 - DNA, 20 bp, synthetic construct
  misc_feature 1..20: note = guide RNA for insertion between UL54 and LORF3 genes of HVT
  source 1..20: mol_type = other DNA
ttagatttccggacagcctg

SEQ ID NO: 6 - DNA, 20 bp, synthetic construct
  misc_feature 1..20: note = guide RNA for insertion into LORF3 of HVT
  source 1..20: mol_type = other DNA
taccacgccgaggcgcctat

(19) United States
(12) Patent Application Publication
(10) Pub. No.: US 2025/0352642 A1
(43) Pub. Date: Nov. 20, 2025

(54) NEW MULTIVALENT HVT VECTOR VACCINE

(71) Applicant: INTERVET INC., Rahway, NJ (US)

(72) Inventors: Martijn Alexander LANGEREIS, Gennep (NL); Iwan VERSTEGEN, Boxmeer (NL); Paul VERMEIJ, St. Anthonis (NL)

(73) Assignee: INTERVET INC., Rahway, NJ (US)

(21) Appl. No.: 18/862,717

(22) PCT Filed: May 4, 2023
(86) PCT No.: PCT/EP2023/061805

§ 371 Date: Nov. 4, 2024

(30) Foreign Application Priority Data
May 5, 2022 (EP) 22171885.1

(51) Int. Cl. A61K 39/255; A61K 39/00; A61P 31/22; C12N 7/00

(52) U.S. Cl. CPC A61K 39/255; A61P 31/22; C12N 7/00; A61K 2039/552; C12N 2710/16321; C12N 2710/16334; C12N 2710/16351

(57) ABSTRACT

The present invention regards a new recombinant HVT (rHVT) construct, useful as multivalent vector vaccine for poultry. The rHVT comprises 4 heterologous genes from poultry pathogens: the VP2 gene from IBDV, the F gene from NDV, and the gD and gI genes from ILTV. The VP2 and F genes are inserted in the Us genome region of the rHVT. The gD-gI genes are inserted in the UL genome region, between the UL54 and the LORF3 genes. The new rHVT-VP2-F-gD-gI proved to be genetically stable in vitro and in vivo, and expressed all inserted genes well. Also it was an effective vaccine against severe challenge infections with NDV, IBDV, and ILTV.

The present invention relates to the field of veterinary vaccines, namely to a vaccine for poultry based on a recombinant herpesvirus of turkeys as viral vector vaccine. In particular the invention relates to a recombinant herpesvirus of turkeys (rHVT), to a host cell comprising said rHVT, to medical uses of said rHVT and said host cell, to vaccines comprising the rHVT and/or the host cell, and to methods for the production of said vaccines.

Recombinant vector viruses are a well-known way to express a heterologous gene and deliver its encoded protein to a human- or non-human animal target. Examples are Vaccinia-, Measles- or Adenovirus vectors. When the heterologous gene encodes an immunogenic protein from a pathogen, this can be a way of effective vaccination of the target against disease caused by that pathogen. As a replicative micro-organism the vector virus can establish a productive infection in a vaccinated target, expressing the heterologous gene along with its own genes, and in this way induce a protective immune-response in the target against the antigen encoded by the heterologous gene.

In veterinary vaccination, and especially for the vaccination of poultry, vector vaccines have gained interest for their relative ease of use and low costs. Several avian vector vaccines have been considered over time, for example based on fowl adenovirus, on fowl pox virus, and in particular on Herpesvirus of turkeys (HVT), see WO 87/04463 and WO 90/002803. An advantage of using HVT as a vector, is that it is non-pathogenic to avians, can carry and express inserted genes, and induces an immunity against pathogenic members of its viral family: Marek's disease virus of serotype 1 or 2 (MDV1 or MDV2).

Over the years genes from different avian pathogens have been inserted in and were expressed by HVT viral vectors, such as from: Newcastle disease virus (NDV), infectious bursal disease virus (IBDV), infectious laryngotracheitis virus (ILTV), and infectious bronchitis virus, see: WO 93/025665; from avian influenza virus (AIV), see WO 2012/052384; or from the parasite Eimeria (Cronenberg et al., 1999, Acta Virol., vol. 43, p. 192-197). This has led to the development of a variety of commercial HVT vector vaccines for poultry, for instance: against ND: Innovax®-ND (MSD Animal Health), and Vectormune® HVT-NDV (Ceva Santé Animale); against ILT: Innovax®-ILT (MSD Animal Health); against IBD: Vaxxitek® HVT+IBD (Boehringer-Ingelheim; previously named: Gallivac™ HVT-IBD), and Vectormune® IBD (Ceva Santé Animale); and against AI: Vectormune® AI (Ceva Santé Animale).

The insertion of a heterologous gene into its viral genome is a burden on a vector virus, as that may affect its replication, expression, and/or its genetic stability, in vitro and/or in vivo. These issues are particularly prominent when more than one heterologous gene is inserted. Such a multivalent recombinant vector vaccine can potentially protect against multiple diseases after a single inoculation. However, such a vector construct must still provide a good replication of the vector and of its inserts, both in vitro and in vivo, and provide an effective expression of all the heterologous genes, at sufficiently high level, and over a significant period of time, to induce and maintain a protective immune-response in a vaccinated target against all intended pathogens.

The genetic stability of a recombinant vector virus will also allow for the extensive rounds of replication in vitro that are necessary for large scale production. In addition, such stability is a requirement to provide compliance with the very high standards of safety and biological stability that must be met by a recombinant virus in vivo (being a genetically modified organism), to be awarded a marketing authorisation from governmental- or regulatory authorities, before it can be introduced into the field as a commercial product.

Many multivalent HVT vector vaccines have been described over time, e.g. as in: WO 93/025665 and WO 96/005291. However, most of the multi-gene constructs described in those publications are only suggested, and only some of the recombinant vectors with multiple inserts were actually constructed and isolated. Very few were ever tested in birds. Overall no results are given on their stability upon replication, or the expression levels of the foreign genes, let alone any data on the induction of an effective immune protection in target animals. It is because of the challenges with the genetic stability and continued expression of the inserts, that only very few multivalent HVT vector constructs have actually made it to become licensed as commercially available vaccine products. Currently these are: Innovax® ND-IBD (MSD Animal Health; WO 2016/102647), Innovax® ND-ILT (MSD Animal Health; WO 2013/057236), ULTIFEND® IBD ND (Ceva Santé Animale; WO 2013/144355), Vaxxitek® HVT+IBD+ND and Vaxxitek® HVT+IBD+ILT (both Boehringer-Ingelheim; WO 2018/112.051).

The efficacy of Innovax® ND-IBD is also described in: van Hulten, M. et al., 2021, Avian Pathol., vol. 50, p. 18-30. The eficacy of Innovax® ND-ILT is described in: Gergen L., et al., 2019, Avian Pathol. vol. 48, p. 45-56.

Tang et al. (2020, Vaccines, vol. 8, p. 97) describe rHVT containing different heterologous genes: an IBDV VP2 gene that is inserted between the HVT UL45 and UL46 genes; the ILTV gD and gI genes inserted between UL65 and UL66; and the AIV HA gene in Us2. These rHVT were only tested in in vitro cell cultures.

WO 2016/102647 describes an HVT vector vaccine wherein the rHVT expresses an IBDV VP2 gene and an NDV F gene, each driven by a separate promoter, in a single expression cassette which is inserted in the Us2 gene of the HVT Us genome region.

WO 2018/112051 describes HVT vector vaccines comprising two genes selected from: IBDV VP2, ILTV gD, and NDV F. The heterologous genes are inserted into the HVT genome, either in the IG1 locus, or between SORF3 and Us2, and are inserted as a single expression cassette which employs a single promoter and whereby the two genes are separated by an IRES or a P2A sequence.

WO 2019/072964 describes a vaccine of a multivalent rHVT vector that is able to protect against MDV, NDV, IBDV, and ILTV. Inserts were placed in the UL54.5 (LORF3) gene and in the Us2 gene of the HVT genome.

However as there are several ways to deal with poultry diseases, there is a constant need for further and improved options for effective vaccination of poultry.

It is therefore an object of the present invention to accommodate to a need in the field, and to provide an rHVT vector vaccine that enables the immunisation of poultry against the 4 avian pathogens: MDV, NDV, IBDV, and ILTV.

Surprisingly it was found that this object can be met, and consequently one or more disadvantages of the prior art can be overcome, by providing an rHVT that expresses the IBDV VP2 and the NDV F genes from the Us genome region, and the ILTV gD and gI genes from the UL genome region.

The inventors attempted to extend the protection already provided by a known rHVT expressing IBDV-VP2- and NDV-F genes (“rHVT-VP2-F”), with a protection against ILTV. The additional heterologous genes selected were the ILTV gD and gI genes. It was found that several multi-insert rHVTs, when tested in vitro and in vivo, did not allow the generation of a stable multivalent recombinant virus: some did not allow the replication of the multivalent recombinant HVT, and some lost the expression of one or more of the heterologous genes.

For example, not successful was the additional insertion of the gD and gI genes into the locus of the HVT UL39 gene (ribonucleotide reductase large subunit), neither when inserted into the central part, nor when inserted into the 3′ region of the UL 39 gene. While these rHVT constructs did replicate in CEF cells in vitro, they completely failed to replicate when inoculated into chickens.

Also ineffective were several constructs comprising the ILTV gD and gI genes inserted in the HVT UL genome region: either between the UL40 (ribonucleotide reductase small subunit) and the UL41 gene (virion host shutoff protein), or inserted between the UL47 gene (tegument phosphoprotein) and the UL48 gene (immediate early gene transactivator): the construct with UL40-41 insert replicated normally in vitro (as compared to the rHVT-VP2-F construct), but at a reduced level in vivo in chicks. In addition, this construct proved to be genetically unstable in vivo, as it lost expression of the F and the VP2 genes. A comparable situation arose for the UL47-48 insert construct, which also replicated normally in vitro and at a reduced rate in vivo, and was also not genetically stable in vivo, as the expression of the gD and gI genes was only at very low level, insufficient for effective vaccination against ILTV. This finding for the UL47-48 insert construct was especially disappointing as the UL47 and UL48 genes had been reported to be non-essential in a transposon gene knock-out study of the HVT genome (Hall et al., 2015, Virology Journal, vol. 12, p. 130).

The inventors' observations were in line with the common perception in this field that more inserts cause more problems to a viral vector's genetic stability in regard to replication and foreign gene expression. In this case it was clear that the fact that the parental vector already expressed two other heterologous genes, complicated things for the additional expression of the ILTV gD and gI genes. This even to such an extent that no predictions of what would be successful multivalent recombinant HVT constructs, could be based on observations in the prior art on what was an allowable insertion site for a heterologous gene in the HVT genome. Similarly, reports of effective replication in vitro of certain rHVT constructs (e.g. as described in Tang et al., 2020, supra) apparently cannot be relied upon to predict in vivo characteristics.

It was therefore unexpected that the additional integration of an expression cassette with ILTV gD and gI genes into a specific insertion site in the HVT UL genome region, did give rise to stable and effective multivalent HVT vector constructs, specifically the insertion of gD and gI between the UL54 and LORF3 genes of HVT. This insertion locus will be referred to herein as the “UL54-LORF3” locus.

The resulting multivalent rHVT vector having F and VP2 inserted in the Us, and the gD and gI genes in the UL54-LORF3 locus, was found to be genetically stable, even after 15 consecutive passages in an in vitro cell culture. The virus was subsequently used for the inoculation of chickens, which accounts for several further replication cycles in vivo. Virus was then re-isolated from the vaccinated chickens at 21 days post vaccination, to determine the level of viremia, and to check for maintained expression of all of the inserted genes. The re-isolated viruses at 21 days p.v. were found to be fully genetically stable: in immuno-fluorescence plaque assay all re-isolated viruses studied demonstrated expression of all the heterologous genes: F, VP2, gD and gI, even at 3 weeks post vaccination.

The vaccinated chickens also showed excellent seroconversion against each of the expressed heterologous antigens: F, VP2, gD, and gI. Antibody levels reached were well above the levels that are known to be required for in vivo protection against infection or disease; details are provided in the Examples.

Surprisingly, the new rHVT vector was also found to be an even more effective vaccine as compared to the rHVT constructs described in patent application no. PCT/EP2021/087445 as HVP412 and HVP 413; those rHVT vectors also have the F and VP2 genes inserted into the Us2 gene, but have the ILTV gD and gI genes inserted between the UL44 and UL45 genes, or between the UL45 and UL46 genes of the HVT genome, respectively. Specifically, the new rHVT vector using the UL54-LORF3 insertion site for ILTV gD and gI, provided a protection against infection and/or disease caused by NDV, IBDV, and ILTV, that was even better on several parameters as that induced by the rHVT vector vaccines HVP412 and HVP413 of '87445.

Therefore, the new multivalent rHVT vector virus is useful as vaccine, in fact: as an improved vaccine, against one, or more, or all of MDV, NDV, IBDV, and ILTV.

The possibility to obtain a vaccination against 4 major poultry diseases from a single vaccine, is hugely beneficial, as it represents an important reduction of stress for the target animals, as well as a reduction of efforts and costs for the poultry farmer.

It is not known exactly how or why an rHVT expressing a VP2- and an F gene can tolerate the additional expression of ILTV gD and gI genes in the UL54-LORF3 insertion locus, whereas insertion in several other sites, that would at first instance appear to be suitable, did not result in stable and effective vector constructs.

Although the inventors do not want to be bound by any theory or model that might explain these findings, they assume that this effect results from the complex interaction of the various expression patterns in the multivalent rHVT, when this is required to replicate and express in vitro and in vivo. For unknown reasons the insertion of the additional genes at this specific locus in the UL results in a multivalent rHVT that has just the right balance between the strength of expression of the heterologous genes, and the strain this puts on the replicative capacity and genetic stability of the HVT itself, whereas other constructs (unpredictably) do not have such balanced composition.

Therefore in one aspect the invention relates to a recombinant herpesvirus of turkeys (rHVT) expressing an infectious bursal disease virus (IBDV) viral protein 2 (VP2) gene and a Newcastle disease virus (NDV) fusion (F) protein gene from a first expression cassette which is inserted in the unique short (Us) region of the genome of the rHVT, characterised in that said rHVT also expresses a glycoprotein D and a glycoprotein I (gD and gI) gene of infectious laryngotracheitis virus (ILTV) from a second expression cassette which is inserted in the unique long (UL) region of the genome of said rHVT, between the UL54 and LORF3 genes.

“Herpesvirus of turkeys (HVT)” is also called MDV3, Meleagrid herpesvirus 1, or turkey herpesvirus. HVT was first described in 1970 (Witter et al., 1970, Am. J. Vet. Res., vol. 31, p. 525). Well-known strains of HVT such as PB1 or FC-126 have for a long time been used as live vaccines for poultry against Marek's disease caused by MDV1 or MDV2.

Herpesvirus of turkeys, Newcastle disease virus, infectious bursal disease virus, and infectious laryngotracheitis virus, are all well-known viruses of veterinary relevance. The same applies to murine- and human cytomegalovirus (mCMV and hCMV), and feline herpesvirus (FHV). Such a virus has the characterising features of its taxonomic group, such as the morphologic, genomic, and biochemical characteristics, as well as the biological characteristics such as physiologic, immunologic, or pathologic behaviour.

General information on these viruses is available e.g. from reference handbooks such as Fields Virology (LWW publ., ISBN: 9781451105636). Information on infection and diseases caused by these viruses is available e.g. from handbooks like: ‘The Merck veterinary manual (2010, 10th ed., 2010, C. M. Kahn edt., ISBN: 091191093X), and: ‘Diseases of poultry’ (2008, 12th ed., Y. Saif ed., Iowa State Univ. press, ISBN-10: 0813807182). Samples of these viruses for use in the invention can be obtained from a variety of sources, e.g. as a field isolate from a human, or from a non-human animal in the wild or on a farm, or from various laboratories, (depository) institutions, or (veterinary) universities. The viruses can be readily identified using routine serological- or molecular biological tools. From all these viruses much genetic information is available digitally in public sequence databases such as NCBI's GenBank™, UniProt, and EMBL's EBI.

As is known in the field, the classification of a micro-organism in a particular taxonomic group is based on a combination of its features. The invention therefore also includes variants of these virus species that are sub-classified therefrom in any way, for instance as a subspecies, strain, isolate, genotype, variant, subtype, or subgroup, and the like.

Further, it will be apparent to a person skilled in the field of the invention that while a particular virus for the invention may currently be assigned to this species, that is a taxonomic classification that could change in time as new insights can lead to reclassification into a new or different taxonomic group.

However, as this does not change the virus itself, or its antigenic repertoire, but only it's scientific name or classification, such re-classified viruses remain within the scope of the invention.

A “recombinant HVT” for the invention is an HVT of which the genetic material has been modified relative to its parental condition, by the molecular insertion of nucleic acids encoding heterologous antigens.

For the invention, a gene or antigen is ‘heterologous’ if it is derived from a (micro-) organism other than an MDV or HVT. Consequently, an rHVT for the invention is not a chimeric viral construct in which a section of the genome has been exchanged by a corresponding section from a related virus, for example as is the case for the ‘novel avian herpesvirus’ as described in WO 1998/037216.

A “VP2 protein gene” is well-known in the art, encoding the IBDV's capsid protein. A VP2 protein gene may be derived from a classic-, or from a variant type IBDV, or may be chimeric.

Similarly, an “F protein gene” is well-known, encoding the NDV's fusion-glycoprotein. For the invention, the F protein gene can be obtained from a lentogenic, mesogenic, or velogenic type of NDV, or may be chimeric.

The term “gene” is used to indicate a section of nucleic acid that is capable of encoding a protein. For the invention this corresponds to an ‘open reading frame’ (ORF), i.e. a protein-encoding section of DNA, from start-to stop codon, not including the gene's promoter. A gene for the invention may encode a complete protein, or may encode a section of a protein, for example encoding only the mature form of a protein, i.e. without a ‘leader’, ‘anchor’, or ‘signal sequence’. A gene may even encode a specific section of a protein, e.g. a section comprising an immunoprotective epitope.

In this regard a “protein” for the invention is a molecular chain of amino acids. The protein can be a native or a mature protein, a pre- or pro-protein, or a functional fragment of a protein. Therefore peptides, oligopeptides and polypeptides are included within the definition of protein, as long as these still contain a relevant immunological epitope and/or a functional region.

For the invention, the term “expression” refers to the well-known principle of gene expression wherein genetic information provides the code for the production of a protein, via transcription and translation.

An “expression cassette” is a nucleic acid fragment comprising at least one heterologous gene and a promoter to drive the transcription of that gene. The termination of the transcription may result from sequences provided by the genomic insertion site of the cassette in the vector genome, or the expression cassette can itself comprise a termination signal, such as a transcription terminator.

In such a cassette, both the promoter and (optionally) the terminator need to be in close proximity to the gene of which they regulate the expression; this is known as being ‘operatively linked’, whereby no significant other sequences are present between them that would intervene with an effective start-respectively termination of the transcription.

While the expression cassette can exist in DNA or in RNA form, because of its intended use in an HVT vector the expression cassette for the invention is employed as DNA. As will be apparent to a skilled person, an expression cassette is a self-contained expression module, therefore its orientation in a vector virus genome is generally not critical.

Optionally the expression cassette may contain further DNA elements, for example to assist with the construction and cloning, such as sites for restriction enzyme recognition or PCR primers.

An expression cassette as a whole is inserted into a single locus in the vector's genome. Different techniques are available to control the locus and the orientation of that insertion. For example by using flanking sections from the genome of the vector, to integrate the cassette by a homologous recombination process in a specific way, e.g. by using overlapping Cosmids as described in U.S. Pat. No. 5,961,982. Alternatively the integration may be done by using the CRISPR/Cas9 technology as described in Tang et al., 2018 (Vaccine, vol. 36, p. 716-722).

For the invention, an “inserted” expression cassette in a vector's genome, refers to the integration into the vector's genomic nucleic acid so that the inserted element gets transcribed and translated along with the vector's native genes. The effect of that insertion on the vector's genome differs depending on the way the insertion is made: the vector genome may become larger, the same, or smaller in size, depending from whether the net result on the genome is an addition, replacement, or deletion of genetic material, respectively. The skilled person is perfectly able to select and implement a certain type of insertion, and make adaptations when needed.

The construction of an expression cassette, and its insertion into an HVT vector, can be done by well-known molecular biological techniques, involving cloning, transfection, recombination, selection, and amplification. These, and other techniques are explained in great detail in standard text-books like Sambrook & Russell: “Molecular cloning: a laboratory manual” (2001, Cold Spring Harbour Laboratory Press; ISBN: 0879695773); Ausubel et al., in: Current Protocols in Molecular Biology (J. Wiley and Sons Inc, NY, 2003, ISBN: 047150338X); C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421).

For the invention, the terms ‘first’ and ‘second’ in regard to the expression cassettes are only used for ease of reference, and not to indicate any order or preference.

The “unique short (Us) region” of the HVT genome is well-known to be the downstream section of the genome between the ‘internal repeat short’, and the ‘terminal repeat short’. The HVT Us is about 8.6 kb in size (see: Kingham et al., 2001, J. of Gen. Virol., vol. 82, p. 1123-1135).

The fully annotated genome sequences of several HVT strains are publicly available e.g. via GenBank, for example: the genome sequence of HVT strain FC-126 is available as GenBank accession number: AF291866, wherein nucleotides 136990-145606 form the Us region.

“ILTV” is also called: Gallid alphaherpesvirus 1. The fully annotated genome sequences of several ILTV strains are publicly available e.g. via GenBank, for example: under accession number NC_006623, for the ILTV reference strain SA-2. The genes for the ILTV gD and gI envelope glycoproteins are located in the Us region of the ILTV genome, as the Us6 and Us7 gene, respectively. The ILTV gD and gI proteins can induce ILTV specific and-protective antibodies, and they are often used in combination. In their natural context these genes partially overlap, whereby the gI gene promoter is located in the upstream gD open reading frame (ORF), and the gD gene terminator is located in the downstream gI ORF. Consequently, when used in combination or when taken from their natural context in an ILT viral genome, the gD and gI genes can conveniently be subcloned as one continuous fragment, e.g. starting with the gD gene promoter and ending after the gI gene.

The “unique long (UL)” region of the HVT genome is the upstream part of the genome, and in HVT is about 110 kb in size. In the genome sequence of HVT strain FC-126 in GenBank accession number: AF291866, the UL region is formed by nucleotides 5910-117777.

The indication “UL54” and similar terms as used herein, is a well-known way in the field of the invention to refer to a specific gene located in the genome of a Herpesvirus, here: in the UL genome region of HVT, see e.g. the annotation applied in GenBank accession number: AF291866. The naming is derived from that of homologous genes in Herpes simplex virus 1, which is the Herpesvirus type species. The same (mutatis mutandis) applies for the other HVT gene indications used herein.

The HVT UL54 gene, also known as the HVT063 gene, encodes a post-translational gene-regulation protein, corresponding to the HSV-1 regulatory protein ICP27.

The HVT gene LORF3, also known as the HVT064 gene, is also called UL54.5, and is sometimes referred to as LORF4, analogous to the naming used in MDV1 and MDV2 for a gene in that location. The HVT LORF3 gene encodes a protein of unknown function which is speculated to correlate with the avian host range of HVT, potentially involving some aspects of lymphotropism.

The term “between” serves to indicate that the inserted second expression cassette is placed in an insertion site (i.e. a locus) on the HVT genome that is in-between and outside of the indicated genes, and in a so-called inter-genic region. Consequently such an insertion is thus not in the open reading frame of the genes it is referring to, e.g. not in the ORF of UL54 or of LORF3. This is thus an essentially different insertion as compared to an intragenic insertion into an ORF, e.g. as in the LORF3 (UL54.5) gene of HVT that is applied in WO 2019/072964.

Details of embodiments and of further aspects of the invention will be described below.

In an embodiment of the rHVT according to the invention, the rHVT is not a chimeric virus.

For the invention, a virus is ‘chimeric’ when a region of its genome is replaced by the corresponding region from the genome of a related virus; e.g. a chimeric HVT would be an HVT in which part of the HVT genome is replaced by the corresponding genome part from an MDV1 or MDV2.

In an embodiment the rHVT according to the invention is characterised in that the first expression cassette comprises in 5′ to 3′ direction and in this order:
a. a murine cytomegalovirus immediate early 1 gene (mCMV-IE1) promoter,
b. an IBDV VP2 gene,
c. a transcription terminator,
d. a human cytomegalovirus immediate early 1 gene (hCMV-IE1) promoter,
e. an NDV F protein gene, and
f. a transcription terminator,
   and whereby the promoters and terminators are operatively linked to the VP2 gene respectively to the F gene.

In this embodiment, the promoter of element a. and the terminator of element c. are operatively linked to the VP2 gene, and the promoter of element d. and the terminator of element f. are operatively linked to the F gene.

The term “comprises” (as well as variations such as “comprising”, “comprise”, and “comprised”) as used herein, intends to refer to all elements, and in any possible combination conceivable for the invention, that are covered by or included in the text section, paragraph, claim, etc., in which this term is used, even if such elements or combinations are not explicitly recited; and not to the exclusion of any of such element(s) or combinations.

Therefore any such text section, paragraph, claim, etc., can therefore also relate to one or more embodiment(s) wherein the term “comprising” (or its variants) is replaced by terms such as “consist of”, “consisting of”, or “consist essentially of”.

The term “in 5′ to 3′ direction”, also known as: ‘in downstream direction’, is well-known in the field. Together with the terms “in this order” it serves to indicate for the invention, the relative orientation which the elements that are summed up thereafter need to have in respect of each other, in order to function with the gene-expression machinery of a host cell in which a rHVT according to the invention can be replicated and expressed. As the skilled person will realise, this direction relates to the DNA strand from the double stranded DNA genome of HVT that is the ‘coding strand’, and it relates to the encoded mRNA molecule that is in the ‘+’ or ‘sense’ orientation.

Nevertheless, and without prejudice to the section above: on the complementary strand of the rHVT ds DNA genome, the ‘template’ strand, the relative order of the listed elements is the same, but on that DNA strand the direction of these elements is 3′ to 5′-, and ‘in upstream direction’.

A “promoter” for the invention is well-known to be a functional region of genetic information that directs the transcription of a downstream coding region. A promoter is thus situated upstream of a gene.

The nomenclature for a promoter is commonly based on the gene of which it controls the expression in its natural context. For example, the term “mCMV-IE1 gene promoter” as used herein, refers to the promoter that in nature drives the expression of the IE1 gene from mCMV, and is thus situated immediately upstream of that gene in the mCMV genome. Because the IE1 gene is a well-documented and clearly recognisable gene, and because the genomes of several mCMV have been sequenced, such a promoter can readily be identified by routine techniques. For example, in a basic protocol a promoter can simply be obtained by roughly subcloning the region between two consecutive genes, e.g. from the stop-codon of an upstream gene to the start-codon of a downstream gene. The promoter can then be identified by standard tests, e.g. by the expression of a marker gene using progressively smaller sections of the cloned region containing a suspected promoter.

Commonly promoters contain a number of recognisable, regulatory regions, such as the enhancer region, which is involved in binding regulatory factors that influence the time, the duration, the conditions, and the level of transcription. While the enhancer region is commonly situated in the upstream part of a promoter, a promoter can also be influenced by regions more downstream towards the start codon that are involved in the binding of transcription factors and directing the RNA polymerase itself. The downstream region of a promoter commonly contains a number of conserved sequence elements such as the TATA box, the CAAT box, and the GC box.

A promoter comprising both the enhancer- and the downstream region is termed a “complete” promoter; a promoter comprising only the downstream region, is termed a “core” promoter.

The mCMV-IE1-gene is well-known in the art, and can readily be obtained from a variety of commercial sources, such as from suppliers of commercial plasmids for cloning and expression. The IE1 gene is also called the ‘major IE gene’ of CMV.

The mCMV-IE1 protein is also called pp89. The mCMV IE1 gene promoter was described in 1985 (K. Dörsch-Häsler, et al., 1985, PNAS, vol. 82, p. 8325). Use of this promoter in heterologous expression is described in WO 87/03.905 and EP 728.842. The nucleotide sequence of the complete mCMV IE gene locus is available e.g. from GenBank under acc. nr. L06816.1. The mCMV itself is available e.g. from the ATCC under acc. nr. VR-1399.

In an embodiment of the rHVT according to the invention, in the first expression cassette the mCMV-IE1gene promoter is a complete promoter, comprising both the core promoter region, as well as the enhancer region for the mCMV-IE1 gene. The complete mCMV-IE1 gene promoter is about 1.4 kb in size.

The term “about” for the invention means ±25% around an indicated value, preferably “about” means ±20, 15, 12, 10, 8, 6, 5, 4, 3, 2% around an indicated value, or even “about” means ±1% around an indicated value, in that order of preference.

In an embodiment the mCMV-IE1 gene promoter for the invention is a DNA molecule of about 1.4 kb, comprising a nucleotide sequence that has at least 95% nucleotide sequence identity to the full length of the region of nucleotides 1-1391 of SEQ ID NO: 1. More preferred is a nucleotide sequence identity of at least 96, 97, 98, or even 99%, in that order of preference.

In an embodiment, the mCMV-IE1 gene promoter is the region of nucleotides 1-1391 of SEQ ID NO: 1.

In an embodiment of the rHVT according to the invention, in the first expression cassette the IBDV VP2 gene for the invention encodes a VP2 protein from an IBDV that is of the classic type. Such genes are well-known and their sequence information is readily available in the prior art, see e.g. GenBank acc.nr: D00869 (strain F52/70), D00499 (strain STC), or AF499929 (strain D78). Alternatively, this gene can be obtained from the genome of a classic IBDV isolated from nature, using routine techniques for manipulating a Birnavirus. Classic type IBDV's can be readily identified using serology, or molecular biology.

As homologs or variants of the IBDV VP2 gene will have equal efficacy and stability, therefore in an embodiment, the IBDV VP2 protein gene for the invention has at least 90% nucleotide sequence identity to the full length of the region of nucleotides 1423-2781 of SEQ ID NO: 1. Preferred is a nucleotide sequence identity of at least 92, 94, 95, 96, 97, 98, or even 99%, in that order of preference.

In an embodiment the IBDV VP2 protein gene for the invention is derived from the classic IBDV strain Faragher 52/70.

In an embodiment the IBDV VP2 protein gene for the invention is the region of nucleotides 1423-2781 of SEQ ID NO: 1.

A “transcription terminator” or terminator, is a regulatory DNA element involved in the termination of the transcription of a coding region into RNA. Commonly such an element encodes a section with a secondary structure, e.g. a hairpin, that can cause the RNA polymerase complex to stop transcription. A transcription terminator is therefore always situated downstream from the stop codon of the region to be translated, thus in the ‘3’ untranslated region' of that ORF. A terminator can also comprise a poly-adenylation (polyA) signal. This provides for the polyadenylation that occurs to most eukaryotic mRNA's, and which plays a role in the transportation and stability of such mRNAs.

For the expression cassettes for the invention, the selection of a specific type of transcription terminator is not critical, as long as effective termination of RNA transcription is provided.

In the first expression cassette for the invention, the transcription terminator element c. between the VP2 and the F genes, not only provides for termination of transcription of the VP2 gene, but also provides for an effective separation of the expression of these genes, by preventing possible read-through of RNA transcription.

The two terminators indicated for the first expression cassette may be the same, or may be different.

In an embodiment of the rHVT according to the invention, the first expression cassette comprises a transcription terminator which comprises both a terminator region and a polyA region.

In an embodiment of the rHVT according to the invention, in the first expression cassette the transcription terminator for the VP2 gene is derived from simian virus 40 (SV40), preferably from the SV40 late gene.

This terminator is available via the commercial ‘pCMVβ’ cloning plasmids (Clontech), since the late 1980's.

In an embodiment of the rHVT according to the invention, in the first expression cassette the transcription terminator for the VP2 gene is derived from the SV40 late gene and is about 0.2 kb in size, and comprises a nucleotide sequence that has at least 95% nucleotide sequence identity to the full length of the region of nucleotides 2812-3021 of SEQ ID NO: 1. More preferred is a nucleotide sequence identity of at least 96, 97, 98, or even 99%, in that order of preference.

In an embodiment, the transcription terminator from the SV40 late gene is the region of nucleotides 2812-3021 of SEQ ID NO: 1.

The hCMV-IE1 gene promoter in its complete version is about 1.5 kb in size and consists of an enhancer, a core promoter, and an intron, whereby the promoter activity proceeds into the intron region, see Koedood et al. (1995, J. of Virol., vol. 69, p. 2194-2207).

An hCMV-IE1 gene promoter can be obtained from the genome of an hCMV virus (which are widely available), by subcloning the genomic area preceding the IE1 gene, using routine molecular biological tools and methods. Alternatively the promoter can be derived for example from commercial expression plasmids, such as pl17, described by Cox et al. (2002, Scand. J. Immunol., vol. 55, p. 14-23), or from commercially available mammalian expression vectors such as the pCMV (Clontech), or the pCMV-MCS series (Stratagene; GenBank acc. nr. AF369966). The genome sequence of hCMV is for example available from GenBank acc. nr. X17403.

From the hCMV-IE1 gene promoter, many highly similar versions are known, e.g. from GenBank. Such homologs and variants are within the scope of the invention.

In an embodiment of the rHVT according to the invention, in the first expression cassette the hCMV-IE1 gene promoter is a core promoter. Such a core promoter will typically be smaller than 1 kb in size; preferably about 0.4 kb in size.

In an embodiment the hCMV-IE1 gene core promoter for the invention is a DNA molecule of about 0.4 kb, comprising a nucleotide sequence that has at least 95% nucleotide sequence identity to the full length of the region of nucleotides 3160-3520 of SEQ ID NO: 1. More preferred is a nucleotide sequence identity of at least 96, 97, 98, or even 99%, in that order of preference.

In an embodiment, the hCMV-IE1 gene core promoter is the region of nucleotides 3160-3520 of SEQ ID NO: 1.

In an embodiment of the rHVT according to the invention, in the first expression cassette the NDV F protein gene is from an NDV that is of the lentogenic type.

Preferably the NDV F protein gene from a lentogenic NDV strain, is from NDV strain Clone 30. NDV Clone 30 is a well-known lentogenic type NDV that has been used for many years as a live vaccine, e.g. as in Nobilis® ND Clone 30 (MSD Animal Health).

In an embodiment, the NDV F protein gene for the invention has at least 90% nucleotide sequence identity to the full length of the region of nucleotides 3545-5206 of SEQ ID NO: 1. Preferably a nucleotide sequence identity of at least 92, 94, 95, 96, 97, 98, or even 99%, in that order of preference.

In an embodiment the NDV F protein gene for the invention is the region of nucleotides 3545-5206 of SEQ ID NO: 1.

In an embodiment of the rHVT according to the invention, in the first expression cassette the transcription terminator for the F gene is derived from the hCMV-IE1 gene. Preferably this transcription terminator is about 0.3 kb in size.

In an embodiment the transcription terminator is derived from the hCMV-IE1 gene, is about 0.3 kb in size, and comprises a nucleotide sequence that has at least 95% nucleotide sequence identity to the full length of the region of nucleotides 5218-5498 of SEQ ID NO: 1. More preferred is a nucleotide sequence identity of at least 96, 97, 98, or even 99%, in that order of preference.

In an embodiment of the rHVT according to the invention, the transcription terminator derived from the hCMV-IE1 gene is the region of nucleotides 5218-5498 of SEQ ID NO: 1.

To optimise the expression of the VP2 and/or of the F gene for the invention, their encoding nucleotide sequence can be subjected to codon optimisation. This is well-known in the art and is commonly applied to improve the expression level of a DNA or RNA sequence in a context that differs from that of the natural origin of the encoded protein. It involves the adaptation of a nucleotide sequence to encode the intended amino acids, but by way of a nucleotide sequence that matches the codon preference (the tRNA repertoire) of the recombinant vector, the host cell, or the target organism in which the sequence will be expressed. Consequently, the nucleotide mutations applied are commonly silent. Such modifications are commonly planned in silico by using one of many computer software programs, after which the desired nucleotide sequence can be synthesized.

Therefore in an embodiment of the rHVT according to the invention, the genes encoding the VP2 and/or the F proteins are codon optimised towards the HVT viral codon preference.

In an embodiment of the rHVT according to the invention, the first expression cassette is an expression cassette as disclosed in WO 2016/102647.

Even more preferably the first expression cassette is the cassette as employed in the rHVT construct described in WO 2016/102647 as HVP360, which is available in the commercial vaccine Innovax® ND-IBD (MSD Animal Health).

An example of a first expression cassette for the invention is presented in SEQ ID NO: 1, the elements of which are described in Table 1.

TABLE 1
Elements of SEQ ID NO: 1

| | |
|---|---|
| Nucleotide region | Elements of SEQ ID NO: 1 |

| | | |
|---|---|---|
| 1 | 1391 | mCMV-IE1 gene promoter + enhancer |
| 1423 | 2781 | IBDV strain F 52/70, VP2 gene |
| 2812 | 3021 | SV40 late gene terminator + polyA signal |
| 3160 | 3520 | hCMV-IE1 gene core promoter |
| 3545 | 5206 | NDV Clone 30 F gene |
| 5218 | 5498 | hCMV IE1 gene terminator |

In an embodiment of the rHVT according to the invention, the first expression cassette for the invention is about 5.5 kb in size.

In an embodiment of the rHVT according to the invention, the first expression cassette for the invention is a DNA molecule of about 5.5 kb, comprising a nucleotide sequence that has at least 95% nucleotide sequence identity to the full length of SEQ ID NO: 1. More preferred is a nucleotide sequence identity of at least 96, 97, 98, or even 99%, in that order of preference.

In an embodiment of the rHVT according to the invention, the first expression cassette for the invention comprises a nucleic acid as depicted in SEQ ID NO: 1. Preferably, the first expression cassette is SEQ ID NO: 1.

As is evident to a skilled person from the composition of the first expression cassette for use in the invention, having two heterologous genes in one large cassette, it is designed and intended for insertion into a single location in the genome of the vector virus.

Also, because an expression cassette is a self-contained expression module, as described, therefore the first expression cassette for the invention can be inserted in the Us genome region of the rHVT according to the invention in different loci and in different orientations.

Several loci of the HVT Us genome region have been demonstrated to allow the insertion of one or more heterologous genes, see e.g. EP 431.668 and WO 2016/102647. For example: in the genes Us2 or Us10, or in the region between Us10 and SORF3, or between Us2 and SORF3.

When inserting in a gene, the consequence is that the normal coding function of the gene receiving the insert is disturbed, or even completely abolished in the resulting rHVT. For Us2 and Us10 this was found not to significantly disturb the replication of the resulting rHVT, nor the expression of the inserted heterologous genes.

Therefore in a preferred embodiment the rHVT according to the invention is characterised in that the first expression cassette is inserted in the Us2 gene, or in the Us10 gene.

In a further preferred embodiment, the rHVT according to the invention is characterised in that the first expression cassette is inserted in the Us2 gene.

The HVT Us2 gene is also known as the HVT088 gene, and refers to the gene in the genome of an HVT virus that correspond to the gene located at nucleotides 140065-140913 in the genome sequence of HVT strain FC-126 in GenBank accession number: AF291866.

In an embodiment the Us2 gene for insertion of the first expression cassette is the Us2 gene of an HVT. Preferably the Us2 gene is from HVT strain FC-126.

The insertion “in the Us2 gene” for the invention, disrupts the function of the Us2 gene. In an embodiment the insertion in Us2 deletes at least 25, 50, or even at least 75% of the Us2 gene, as described for HVP360 in WO 2016/102647.

To facilitate the convenient construction, manipulation, and insertion of an expression cassette into an HVT for the invention, the cassette can itself be comprised in a DNA molecule, such as a vehicle allowing cloning or transfection, e.g. such as a plasmid, a Cosmid, a Bacmid, etc., see WO 93/025665 and EP 996.738. Examples of common cloning plasmids are e.g. plasmids from the pBR322 or the pUC series. These are widely commercially available.

A plasmid comprising an expression cassette is commonly referred to as a ‘transfervector’, ‘shuttle vector’, or ‘donor plasmid’. In this situation the plasmid comprises an expression cassette with flanking sequence regions from the target insertion locus of the vector's genome, to direct the insertion.

Typically, a transfervector that is used in transfection is not itself integrated into the genome of the vector, it only facilitates the integration of the expression cassette it carries, e.g. by allowing the insertion to occur by homologous recombination. Consequently, in the case of the first expression cassette for use in the present invention, the first expression cassette is preferably flanked at its 5′ and its 3′ ends by sections of the Us2 gene of HVT, which direct the process of insertion of that cassette into Us2.

Therefore, in an embodiment of the rHVT according to the invention, the first expression cassette is flanked by sequences from the Us2 gene of HVT.

Preferably the flanking Us2 gene sequences are nucleotides 140143-140541 from GenBank accession nr. AF291866, and nucleotides 140541-141059 from GenBank accession nr. AF291866.

As described above, the present rHVT vector advantageously comprises a further set of heterologous genes by way of a second expression cassette, which genes are stably maintained in the rHVT during replication and expression, both in vitro and in vivo.

Therefore, in an embodiment the rHVT according to the invention is characterised in that the second expression cassette comprises in 5′ to 3′ direction and in this order:
a. an ILTV gD gene with an upstream promoter and a downstream terminator, and
b. an ILTV gI gene with an upstream promoter and a downstream terminator, and whereby the promoters and terminators are operatively linked to the gD gene respectively to the gI gene.

In an embodiment of the rHVT according to the invention, in the second expression cassette the section of the second cassette with the gD gene with promoter and terminator, and the gI gene with promoter, is taken as a whole from an ILTV genome. As described above: because of the overlap of the ILTV gD and gI genes in their natural context, the gD gene terminator and the gI gene promoter are then comprised in the gI gene and in the gD gene respectively.

SEQ ID NO: 2 presents an example of the nucleotide sequence of a second expression cassette for the present invention, as a Hind3 fragment of about 3.2 kb; the elements of which are described in Table 2.

TABLE 2
Elements of SEQ ID NO: 2

| | |
|---|---|
| Nucleotide region | Elements of SEQ ID NO: 2 |

| | | |
|---|---|---|
| 13 | 580 | ILTV gD gene promoter |
| 581 | 1885 | ILTV gD gene |
| 1993 | 3081 | ILTV gI gene |
| 3097 | 3151 | FHV1 Us9 gene transcription terminator |

Preferably, the section of the gD gene with promoter and terminator, and the gI gene with promoter, is formed by a DNA molecule of about 3 kb, comprising a nucleotide sequence that has at least 90% nucleotide sequence identity to the full length of the region of nucleotides 13-3081 of SEQ ID NO: 2. Even more preferred is a nucleotide sequence identity of at least 92, 94, 95, 96, 97, 98, or even 99%, in that order of preference.

In an embodiment of the rHVT according to the invention, in the second expression cassette, the section comprising the gD gene with promoter and terminator and the gI gene with promoter, is formed by the region of nucleotides 13-3081 of SEQ ID NO: 2.

In an embodiment of the rHVT according to the invention, in the second expression cassette the transcription terminator of the gI gene is derived from FHV1, preferably from the FHV1 Us9 gene. An FHV1 Us9 gene is for example disclosed in GenBank accession number D42113.

In an embodiment of the rHVT according to the invention, in the second expression cassette the transcription terminator is derived from the FHV1 Us9 gene and is about 0.05 kb in size, and comprises a nucleotide sequence that has at least 95% nucleotide sequence identity to the full length of the region of nucleotides 3097-3151 of SEQ ID NO: 2. More preferred is a nucleotide sequence identity of at least 96, 97, 98, or even 99%, in that order of preference.

In an embodiment, the transcription terminator from the FHV1 Us9 gene is the region of nucleotides 3097-3151 of SEQ ID NO: 2.

In an embodiment of the rHVT according to the invention, the second expression cassette for the invention is about 3.2 kb in size.

In an embodiment of the rHVT according to the invention, the second expression cassette for the invention is a DNA molecule of about 3.2 kb, comprising a nucleotide sequence that has at least 95% nucleotide sequence identity to the full length of SEQ ID NO: 2. More preferred is a nucleotide sequence identity of at least 96, 97, 98, or even 99%, in that order of preference.

In an embodiment of the rHVT according to the invention, the second expression cassette for the invention comprises a nucleic acid as depicted in SEQ ID NO: 2. Preferably, the second expression cassette is SEQ ID NO: 2.

As described, the second expression cassette for the invention, comprising the ILTV gD and gI genes, is inserted in the intergenic region between the ORFs of the HVT genes UL54 and LORF3. Because these two genes in the HVT genome are situated in so-called tail-to-tail orientation (their respective reading directions pointing towards each other), therefore the intergenic region between UL54 and LORF3 in HVT is formed by the region that is downstream of the UL54 stop codon and downstream of the LORF3 stop codon; this region in HVT is about 0.9 kb in size. FIG. 1 herein, graphically depicts these orientations.

Therefore in a preferred embodiment of the rHVT according to the invention, the second expression cassette for the invention is inserted in the HVT genomic region of the nucleotides 110.383-111.302, as published in GenBank accession nr. AF291866. More preferably the second expression cassette for the invention is inserted in the HVT genome between the nucleotides 110.395 and 110.396, as published in GenBank accession nr. AF291866.

The rHVT according to the invention is preferably based on a parental HVT that is an established HVT vaccine strain, which replicates well, and is known to be suitable for inoculation of young birds or bird embryos in ovo; for example the HVT vaccine strains PB1 or FC-126. These are generally available: FC-126 from ATCC: VR #584-C, and PB1 is commercially available as live vaccine in frozen infected cells, e.g. from MSD Animal Health as: Nobilis® Marek THV Lyo.

The incorporation of the first and second expression cassettes, both as defined herein for the invention, do not increase the virulence or pathogenicity of the parental HVT (on the contrary), and no reversion to virulence is to be expected, as HVT are naturally apathogenic.

Therefore, in an embodiment the parental HVT used for generation of the rHVT according to the invention is an HVT vaccine strain; preferably an HVT vaccine strain of the PB1- or of the FC-126 strain.

The rHVT according to the invention is a live recombinant carrier micro-organism, or a “vector” virus, which can advantageously be used for vaccination of poultry. It combines the features of being a safe and effective vaccine against Marek's disease (MD), and against one or more or all of: infectious bursal disease (IBD), Newcastle disease (ND), and infectious laryngotracheitis (ILT), and in addition is genetically stable.

Being “genetically stable” for the invention means that the genetic make-up of the rHVT according to the invention does not change in subsequent rounds of virus replication. In the alternative, unstable constructs can lead to inefficient viral replication, and/or to the loss of expression of one or more of the inserted heterologous gene(s), which may lead to over-replication of the defective virus mutant. This stability can conveniently be monitored with routine techniques, e.g. by subjecting the rHVT according to the invention to subsequent passaging in cell culture. Virus re-isolated during these steps, can be plated on cell culture dishes, can optionally be covered with agar, and incubated until HVT-specific plaques become visible; all using routine techniques. Next the plaques can be stained for expression of the VP2, F, or the gD and gI proteins using suitable antibody preparations in an immunofluorescence assay (IFA) protocol, with adequate positive and negative controls. Any plaques that do no longer show fluorescence for a particular heterologous protein can then be recorded, whereby preferably about 100 individual plaques from a particular rHVT sample should be monitored.

It was surprisingly found that the rHVT according to the invention maintained the presence and the expression of each of the VP2, F, gD and gI protein genes, in all of the plaques tested, even after 15 consecutive cell-culture passages, and subsequently 21 days of replication in vivo. Details are described in the Examples.

This is a strong and highly significant improvement over many of the alleged multivalent HVT vector constructs that are described in the prior art, but without demonstrated stability in vitro and in vivo.

Also, considering that all of the VP2, F, gD and gI genes have already been employed as insert in effective HVT vector vaccines, the fact that they are stably maintained and expressed is also credible proof that the rHVT according to the invention will induce a protective immune response in poultry against the pathogens from which these antigens were derived, as well as against MDV, and thus will be an effective multivalent vector vaccine.

Therefore, in an embodiment of the rHVT according to the invention, one or more of the conditions apply selected from the groups consisting of:
for the first expression cassette:
   the mCMV-IE1 gene promoter is a complete promoter;
   the IBDV VP2 gene encodes a VP2 protein from a classic type IBDV;
   the transcription terminator for the VP2 gene comprises both a terminator region and a polyA region; preferably the transcription terminator is derived from SV40;
   the hCMV-IE1 gene promoter is a core promoter;
   the NDV F gene is from a lentogenic NDV strain, preferably from NDV strain Clone 30;
   the transcription terminator for the F gene is derived from the hCMV-IE1 gene;
   the genes encoding the VP2 and/or the F proteins are codon optimised towards the HVT viral codon preference;
   the first expression cassette is an expression cassette as disclosed in WO 2016/102647; even more preferably the first expression cassette is the cassette as employed in the rHVT construct described in WO 2016/102647 as HVP360;
   the first expression cassette for the invention is a DNA molecule of about 5.5 kb, comprising a nucleotide sequence that has at least 95% nucleotide sequence identity to the full length of SEQ ID NO: 1; more preferred is a nucleotide sequence identity of at least 96, 97, 98, or even 99%, in that order of preference;
   the first expression cassette for the invention comprises a nucleic acid as depicted in SEQ ID NO: 1; preferably, the first expression cassette is SEQ ID NO: 1;
and/or for the second expression cassette:
   the second expression cassette comprises in 5′ to 3′ direction and in this order:
      an ILTV gD gene with an upstream promoter and a downstream terminator, and
      an ILTV gI gene with an upstream promoter and a downstream terminator, and whereby the promoters and terminators are operatively linked to the gD gene respectively to the gI gene;
   the promoter may be a core promoter or may be a complete promoter;
   the genes encoding the gD and/or the gI gene are codon optimised towards the HVT viral codon preference;
   the promoter for the gD gene is an ILTV gD gene promoter;
   the promoter for the gI gene is an ILTV gI gene promoter;
   the terminator for the gD gene is an ILTV gD gene terminator;
   the terminator for the gI gene is an ILTV gI gene terminator;
   the second expression cassette is a DNA molecule of about 3.2 kb, comprising a nucleotide sequence that has at least 95% nucleotide sequence identity to the full length of SEQ ID NO: 2; more preferred is a nucleotide sequence identity of at least 96, 97, 98, or even 99%, in that order of preference;
   the second expression cassette comprises a nucleic acid as depicted in SEQ ID NO: 2; preferably, the second expression cassette is SEQ ID NO: 2;
   the second expression cassette is inserted in the HVT genomic region of the nucleotides 110.383-111.302, as published in GenBank accession nr. AF291866; more preferably the second expression cassette for the invention is inserted in the HVT genome between the nucleotides 110.395 and 110.396, as published in GenBank accession nr. AF291866;
and/or for the rHVT:
   the first expression cassette is inserted in the Us2 gene, or in the Us10 gene; preferably the first expression cassette is inserted in the Us2 gene;
   the Us2 gene for insertion of the first expression cassette is the Us2 gene of an HVT; preferably the Us2 gene is from HVT strain FC-126;
   the parental HVT used for generation of the rHVT is an HVT vaccine strain; preferably the parental HVT is an HVT vaccine strain of the PB1- or of the FC-126 strain.

By different methods of cloning and transfection, the first and second expression cassettes for the invention can be used to obtain the rHVT according to the invention, stably comprising and expressing the expression cassettes in its genome as described herein.

Therefore, a further aspect of the invention relates to a method for the construction of the rHVT according to the invention, said method comprising the insertion of the first and the second expression cassettes as defined for the invention, into a region of the genome of an HVT as defined for the invention.

The insertion of an expression cassette according to the invention into an HVT genome to generate the rHVT according to the invention, can be performed in different ways, all known in the art. One convenient way is to use a transfervector and the technique of homologous recombination, as described above.

Alternatively the rHVT according to the invention can be generated using the CRISPR/Cas9 technology; for example as described by Tang et al., 2018 (supra).

In particular an rHVT-VP2-F vector virus can be used, which is available since 2017 in the commercial vaccine Innovax ND-IBD. This can be further manipulated by inserting the second expression cassette as described herein into the UL 54-LORF3 locus of the genome of the rHVT as described herein, using the CRISPR/Cas9 technology. The specific guide RNA sequences that can be used to aim this insert to this locus are described in the Examples.

The rHVT according to the invention can be amplified by common techniques, preferably by replication in vitro, e.g. in cultures of avian cells, typically primary chicken embryo fibroblast cells (CEF's). These can be prepared by trypsinisation of chicken embryos, all well-known in the art. The CEF's are plated in monolayers and infected with the rHVT. This process can be scaled up to industrial size production.

Commonly the rHVT are collected by harvesting the infected host cells that contain the rHVT in a cell-associated form. These cells are then taken up in an appropriate carrier composition to provide stabilisation during freezing and storage. Next the infected cells are commonly filled into glass ampoules, which are sealed, frozen, and stored in liquid nitrogen. Upon use for vaccination, the ampoules are thawed, and the infected cells are taken up into a suitable dilution buffer for in-use stabilisation. In a preferred embodiment, the dilution buffer is a buffer as disclosed in WO 2019/121888.

Although cell-associated frozen storage of HVT is preferred, in situations where use of liquid nitrogen is not feasible, an alternative is to use freeze-drying: this employs the favourable characteristic of HVT that it can be isolated from its host cell by cell-disruption, e.g. by French press or sonifier, using the whole culture. This can be clarified by centrifugation, and is then taken up into a stabiliser, and freeze-dried for prolonged storage.

Therefore, in a further aspect, the invention relates to a host cell comprising the rHVT according to the invention.

A “host cell” for the invention, is a cell that is susceptible to infection and replication by an HVT. Examples of such cells are avian cells, and in particular lymphocytes or fibroblasts.

Preferably the host cell according to the invention is a host cell kept under in vitro conditions.

In an embodiment, the host cell according to the invention is a primary avian cell; i.e. a cell that is derived in vitro from a non-human animal tissue or -organ, and not from an immortalised cell-line. Typically primary cells can only perform a small and limited number of cell-divisions.

In an embodiment the primary avian host cell for the invention is a primary chicken embryo fibroblast (CEF).

In an embodiment, the host cell according to the invention is an immortalised avian cell. Several immortalised avian cell-lines have been described, for example in WO 97/044443 and WO 98/006824.

In a preferred embodiment the immortalised avian host cell according to the invention is an immortalised CEF; preferably an immortalised CEF as disclosed in WO 2016/087560.

As described, the main advantageous use of the rHVT according to the invention is in a vaccine for poultry, providing a safe, stable and effective vaccination against MD, IBD, ND and/or ILT or associated signs of disease, and can be administered to poultry at a very young age.

Therefore, a further aspect of the invention relates to the rHVT according to the invention, and/or to the host cell according to the invention, for use in a vaccine for poultry.

Different ways of a ‘use in a vaccine’ of the rHVT or of the host cell, both according to the invention, have been outlined above, and comprise the use as cell-free virus or as cell-associated virus in a host cell, in a vaccine composition for inoculation of poultry.

Also, in a further aspect the invention relates to a vaccine for poultry comprising the rHVT according to the invention and/or the host cell according to the invention, and a pharmaceutically acceptable carrier.

A “vaccine” is well-known to be a composition comprising an immunologically active compound, in a pharmaceutically acceptable carrier. The ‘immunologically active compound’, or ‘antigen’ is a molecule that is recognised by the immune system of the inoculated target and induces a protective immunological response from the humoral- and/or the cellular immune system of the target.

The vaccine according to the invention provides protection of poultry against infection and/or disease caused by MDV, IBDV, NDV, and/or ILT. This effect is obtained by preventing or reducing the establishment or the proliferation of a productive infection by one or more of these viruses, in their respective target organs. This is achieved for example by reducing the viral load or shortening the duration of the viral replication. In turn this leads to a reduction in the target animal of the number, the intensity, or the severity of lesions and associated clinical signs of disease caused by the viral infection.

However, depending on the virulence of the MDV, IBDV, NDV, or ILTV field virus that is prevalent in a certain poultry farm or in a certain area, it may be necessary to add a further vaccine component of one or more of these viruses, to assure effective vaccination for pathogenic- or serologic variants of these viruses. This is all well-known in the art.

The determination of the effectiveness of a use as a vaccine for poultry, or of a vaccine for poultry, both according to the invention, is well within the skills of the routine practitioner, and can be done for instance by monitoring the immunological response following vaccination, or by testing the appearance of clinical symptoms or mortality after a challenge infection, e.g. by monitoring the targets' signs of disease, clinical scores, serological parameters, or by re-isolation of the challenge pathogen, and comparing these results to a vaccination-challenge response seen in unvaccinated animals. Different ways to assess each of the four virus-infections are well-known in the art.

The protection against MD, IBD, ND, and ILT induced by the use, the vaccine, or by the vaccination, all according to the invention, results in the vaccinated targets in an improvement of health and economic performance. This can for instance be assessed from parameters such as increase of one or more of: survival, growth rate, feed conversion, egg-production, and number- and health of offspring. Further effects are reduced costs for health care, and increased economy of operation.

Various embodiments, preferences and examples of the use, the vaccine, or the vaccination, all according to the invention, will be outlined below.

The term “poultry” for the invention relates to a species of bird of relevance to veterinary practice, and that is susceptible to inoculation with HVT; the preferred poultry species are: chicken, turkey, goose, duck, and quail. Chickens are the most preferred species.

For the invention, the poultry may be of any type, breed, or variety, such as: layers, breeders, broilers, combination breeds, or parental lines of any of such type. Preferred types are: broiler, breeder, and layer. Most preferred are broiler and layer type poultry.

A “pharmaceutically acceptable carrier” is intended to aid in the stabilisation and administration of the vaccine, while being harmless and well-tolerated by the target. Such a carrier can for instance be sterile water or a sterile physiological salt solution. In a more complex form the carrier can e.g. be a buffer, which can comprise further additives, such as stabilisers or conservatives. Details and examples are for instance described in well-known handbooks such as: “Remington: the science and practice of pharmacy” (2000, Lippincott, USA, ISBN: 683306472), and: “Veterinary vaccinology” (P. Pastoret et al. ed., 1997, Elsevier, Amsterdam, ISBN 0444819681).

For the present invention, and when the vaccine is in the form of cell-associated HVT, then the pharmaceutically acceptable carrier is preferably a mixture of culture medium, about 10% v/v serum, and about 6% v/v DMSO. This carrier also provides for the stabilisation of the rHVT-infected host cells during freezing and frozen storage. The serum can be any serum routinely used for cell culturing such as foetal- or new-born calf serum.

The vaccine according to the invention is prepared from an rHVT according to the invention by methods as described herein, which are readily applicable by a person skilled in the art. For example, the rHVT according to the invention is constructed by insertion of the expression cassettes as described for the invention by transfection and recombination. Next the desired rHVT is selected, and is amplified industrially in smaller or larger volumes, preferably in in vitro cell cultures, e.g. in CEF's. From such cultures a suspension of host cells infected with the rHVT is harvested, either as whole infected cells or as a cell-free preparation obtained by cell-disruption. This suspension is formulated into a vaccine with a suitable pharmaceutical carrier, and the final product is packaged. Cell-associated vaccine is then stored in liquid nitrogen, and freeze-dried vaccine at −20 or at +4° C.

General techniques and considerations that apply to the manufacture of vaccines under well-known standards for pharmaceutical production are described for instance in governmental directives and regulations (Pharmacopoeia, 9CFR) and in well-known handbooks (“Veterinary vaccinology” and: “Remington”, both supra). Commonly such vaccines are prepared sterile, and are prepared using excipients of pharmaceutical quality grade.

Such preparations will incorporate microbiological tests for sterility, and absence of extraneous agents; and may include studies in vivo or in vitro for confirming efficacy and safety. After completion of the testing for quality, quantity, sterility, safety, and efficacy, the vaccine can be released for sale. All these are well-known to a skilled person.

In an embodiment the vaccine for poultry according to the invention is a cell-associated vaccine.

“Cell-associated” means that the rHVT according to the invention is comprised in host cells in vitro, according to the invention. Consequently a vaccine of this type comprises both the host cells as well as the rHVT, both according to the invention.

The target animal for the vaccine according to the invention can in principle be healthy or diseased, and may be positive or negative for presence of MDV, IBDV, NDV, or ILTV, or for antibodies against MDV, IBDV, NDV, or ILTV. Also the target can be of any weight, sex, or age at which it is susceptible to the vaccination. However it is evidently favourable to vaccinate healthy, uninfected targets, and to vaccinate as early as possible to prevent any field infection and its consequences.

A vaccine according to the invention can thus be used either as a prophylactic- or as a therapeutic treatment, or both, as it interferes both with the establishment and with the progression of an infection by MDV, IBDV, NDV, or ILTV.

In that respect, a further advantageous effect of the reduction of viral load by the vaccine according to the invention, is the prevention or reduction of shedding and thereby the spread of field virus, both vertically to offspring, and horizontally within a flock or population, and within a geographical area. Consequently, the use of a vaccine according to the invention leads to a reduction of the prevalence of MDV, IBDV, NDV or ILTV.

Therefore further aspects of the invention are:
a use of a vaccine for poultry according to the invention for reducing the prevalence of MDV, IBDV, NDV, or ILTV in a population or in a geographical area, and
the vaccine for poultry according to the invention for reducing the prevalence of MDV, IBDV, NDV, or ILTV in a population or in a geographical area.

The vaccine according to the invention already provides a multivalent immunity: against IBD, ND, and ILT by the expression of the heterologous inserts, and in addition against MD by the HVT vector itself. Nevertheless, it can be advantageous to make further combinations with additional immunoactive components. This can serve to enhance the immune protection already provided, or to expand it to other pathogens.

Therefore, in an embodiment, the vaccine according to the invention is comprising at least one additional immunoactive component.

Such an “additional immunoactive component” may be an antigen, an immune enhancing substance, a cytokine, a further vaccine, or any combination thereof. This provides advantages in terms of cost, efficiency, and animal welfare. Alternatively, the vaccine according to the invention, may itself be added to a vaccine.

In an embodiment the at least one additional immunoactive component is an immunostimulatory compound; preferably a cytokine or an immunostimulatory oligodeoxynucleotide.

The immunostimulatory oligodeoxynucleotide is preferably an immunostimulatory non-methylated CpG-containing oligodeoxynucleotide (INO). A preferred INO is an avian Toll-like receptor (TLR) 21 agonist, such as described in WO 2012/089800 (X4 family), WO 2012/160183 (X43 family), or WO 2012/160184 (X23 family).

In an embodiment the at least one additional immunoactive component is an antigen which is derived from a micro-organism pathogenic to poultry. This antigen can be ‘derived’ in any suitable way, for instance as a ‘live’ attenuated-, an inactivated-, or a subunit antigen from that micro-organism pathogenic to poultry.

The additional antigen derived from a micro-organism pathogenic to poultry, is preferably derived from one or more micro-organisms selected from the following groups consisting of:
viruses: infectious bronchitis virus, NDV, Adenovirus, avian influenza virus, Egg drop syndrome virus, IBDV, chicken anaemia virus, avian encephalo-myelitis virus, fowl pox virus, turkey rhinotracheitis virus, duck plague virus (duck viral enteritis), pigeon pox virus, MDV, avian leucosis virus, ILTV, avian pneumovirus, and Reovirus;
bacteria: Escherichia coli, Salmonella, Ornitobacterium rhinotracheale, Haemophilus paragallinarum, Pasteurella multocida, Erysipelothrix rhusiopathiae, Erysipelas, Mycoplasma, and Clostridium;
parasites: Eimeria; and
fungi: Aspergillus.

The additional antigen may also be a further vector vaccine, e.g. based on HVT, on MDV2, on NDV, etcetera.

In an embodiment of the vaccine according to the invention, the additional antigen derived from a micro-organism pathogenic to poultry is a ‘live’ attenuated vaccine strain of MDV, IBDV, NDV, or ILTV. This serves to improve and expand the immunogenicity of the vaccine according to the invention, and this is advantageous in those cases or geographic areas where very virulent field strains of MDV, IBDV, NDV, or ILTV are prevalent.

In this regard, the combination of an HVT with an MDV1, MDV2, or HVT is known; for the invention an MDV of strain Rispens (MDV1), or strain SB1 (MDV2), or an HVT of strains FC-126 or PB1 is preferred as additional immunoactive component.

To improve the response against ND, the rHVT according to the invention may be combined with an NDV vaccine strain such as the mild live NDV vaccine strain C2.

Similarly, to improve the response against IBD, the rHVT according to the invention may be combined with a live IBDV vaccine strains such as D78, PBG98, Cu-1, ST-12, or 89-03.

As the skilled person will appreciate, these ‘combinations’ also include vaccination schedules wherein the rHVT according to the invention and the additional immunoactive component are not applied combined or simultaneous, but in a concurrent- or sequential vaccination schedule; e.g. the rHVT may be applied in ovo, the NDV C2 at day one, and the IBDV 89-03 at about day 17 of age.

Therefore, in an embodiment of the vaccine according to the invention comprising at least one additional immunoactive component, the at least one additional immunoactive component is a micro-organism selected from the group consisting of a vaccine strain from: MDV, IBDV, NDV, or ILTV, or any combination thereof.

More preferably the additional immunoactive component is one or more selected from the group consisting of: MDV Rispens, MDV SB1, NDV C2, IBDV D78, and IBDV 89-03.

A vaccine according to the invention can be prepared by methods as described and exemplified herein.

Therefore, a further aspect of the invention relates to a method for the preparation of the vaccine for poultry according to the invention, said method comprising the steps of:
a. infecting host cells in vitro with the rHVT according to the invention,
b. culturing and harvesting the infected host cells, and
c. admixing the harvested infected host cells with a pharmaceutically acceptable carrier.

Suitable host cells and pharmaceutically acceptable carriers for the invention have been described above. Also, suitable in vitro methods for infection, culture, and harvesting are well-known in the art and are described and exemplified herein.

Consequently, the different aspects and embodiments of the invention can advantageously be used to produce a safe, stable, and effective vaccine for poultry according to the invention.

Therefore, in a further aspect, the invention relates to the use of the rHVT, and/or of the host cell, both according to the invention, for the manufacture of a vaccine for poultry.

It goes without saying that admixing other compounds, such as stabilisers, carriers, adjuvants, diluents, emulsions, and the like to vaccines according to the invention are also within the scope of the invention. Such additives are described in well-known handbooks such as: “Remington”, and “Veterinary Vaccinology” (both supra).

This way the efficacy of a vaccine according to the invention, to protect poultry with a single inoculation at very young age against MD, IBD, ND, and ILT can be further optimised when needed.

A vaccine according to the invention can be prepared in a form that is suitable for administration to a poultry target, and that matches with a desired route of application, and with the desired effect.

Depending on the route of application of the vaccine according to the invention, it may be necessary to adapt the vaccine's composition. This is well within the capabilities of a skilled person, and generally involves the fine-tuning of the efficacy or the safety of the vaccine. This can be done by adapting the vaccine dose, quantity, frequency, route, by using the vaccine in another form or formulation, or by adapting the other constituents of the vaccine (e.g. a stabiliser or an adjuvant).

The vaccine according to the invention in principle can be given to target poultry by different routes of application, and at different points in their lifetime, provided the inoculated rHVT can establish a protective infection.

However, because an infection with MDV, IBDV, NDV, or ILTV can be established already at very young age, it is advantageous to apply the vaccine according to the invention as early as possible. Therefore, the vaccine according to the invention can be e.g. applied at the day of hatch (“day one”), or in ovo, at about 18 days of embryonic development, all well-known in the art.

Therefore, in an embodiment, the vaccine according to the invention is administered to poultry in ovo.

Equipment for automated injection of a vaccine into a fertilized egg at industrial scale, is available commercially, e.g. from Embrextm, or Vinovotm. This provides the earliest possible protection, while minimising labour costs. Different in ovo inoculation routes are known, such as into the yolk sac, the embryo, or the allantoic fluid cavity; these can be optimised as required. Preferably in ovo inoculation with an HVT is performed such that the needle touches the embryo.

Preferably a vaccine according to the invention is formulated as an injectable liquid, suitable for injection, either in ovo, or parenteral; for example as: a suspension, solution, dispersion, or emulsion.

In an embodiment, the vaccine according to the invention is administered by parenteral route. Preferably by intramuscular- or subcutaneous route.

The exact amount of rHVT according to the invention, per animal dose of the vaccine according to the invention, is not as critical as it would be for an inactivated- or subunit type vaccine; this is because the rHVT will replicate in the target animal up to a level of viremia that is biologically sustainable. In principle the vaccine dose only needs to be sufficient to initiate such a productive infection. A higher inoculum dose hardly shortens the time it takes to reach an optimal viraemic infection in the host. Therefore, very high doses are not more effective and in addition are not attractive for economic reasons.

A preferred inoculum dose is therefore between 1×10\hat{ }1 and 1×10\hat{ }5 plaque forming units (pfu) of rHVT according to the invention per animal dose; more preferably between 1×10\hat{ }2 and 1×10\hat{ }4 pfu/dose; even more preferably between 500 and 5000 pfu/dose; most preferably between about 1000 and about 3000 pfu/dose.

When the vaccine according to the invention is cell-associated, these amounts of rHVT are comprised in infected host cells. Methods to count viral particles of the rHVT according to the invention are well-known.

The volume per animal dose of the rHVT according to the invention can be optimised according to the intended route of application: in ovo inoculation is commonly applied with a dose of between about 0.01 and about 0.5 ml/egg, and parenteral injection is commonly done with a dose of between about 0.1 and about 1 ml/bird.

Determination of what is an immunologically effective amount of the vaccine according to the invention, or the optimisation of the vaccine's volume per animal dose, are both well within the capabilities of the skilled artisan.

The dosing regimen for applying the vaccine according to the invention to a target organism can be in single- or in multiple doses, in a manner compatible with the formulation of the vaccine, and in such an amount as will be immunologically effective.

Preferably, the regimen for the administration of a vaccine according to the invention is integrated into existing vaccination schedules of other vaccines that the target poultry may require, in order to reduce stress to the animals and to reduce labour costs. These other vaccines can be administered in a simultaneous, concurrent, or sequential fashion, in a manner compatible with their licensed use.

As described above, and as exemplified hereinafter, the vaccine according to the invention can advantageously be used to prevent or reduce infection by one, or more, or all of MDV, IBDV, NDV, and ILTV, and the prevention or reduction of the (signs of) disease associated with such infections, by a single inoculation at very young age.

Therefore, further aspects of the invention are:
a use of the vaccine for poultry according to the invention, for preventing or reducing infection by one or more of MDV, IBDV, NDV, and ILTV, and/or their associated signs of disease.
a method for preventing or reducing infection by one or more of MDV, IBDV, NDV, and ILTV, and/or their associated signs of disease, the method comprising the administration of the vaccine according to the invention to poultry.
a method of vaccination of poultry to prevent or reduce infection by one or more of MDV, IBDV, NDV, and ILTV, and/or their associated signs of disease, the method comprising the step of inoculating said poultry with the vaccine according to the invention.

Details on the use of the vaccine according to the invention, by inoculation of poultry have been described above; specifically the inoculation by intramuscular or subcutaneous inoculation of day old chicks, and the in ovo inoculation of 18 day old embryos.

The invention will now be further described with reference to the following, non-limiting, examples.

### Sequence Listing in ST25 Format

SEQUENCE LISTING

| | |
|---|---|
| <110> | Intervet International BV |
| <120> | New multivalent HVT vector vaccine |
| <130> | 25404 |
| <160> | 6 |
| <170> | PatentIn version 3.5 |
| <210> | 1 |
| <211> | 5498 |
| <212> | DNA |
| <213> | Artificial Sequence |
| <220> | |
| <223> | Expression cassette with IBDV-VP2 and NDV-F genes |
| <400> | 1 |

| | |
|---|---|
| aactccgccc gttttatgac tagaaccaat agtttttaat gccaaatgca ctgaaatccc | 60 |
| ctaatttgca aagccaaacg ccccctatgt gagtaatacg gggacttttt acccaatttc | 120 |
| ccaagcggaa agccccctaa tacactcata tggcatatga atcagcacgg tcatgcactc | 180 |
| taatggcggc ccatagggac tttccacata gggggcgttc accatttccc agcatagggg | 240 |
| tggtgactca atggccttta cccaagtaca ttgggtcaat gggaggtaag ccaatgggtt | 300 |
| tttcccatta ctggcaagca cactgagtca aatgggactt tccactgggt tttgcccaag | 360 |
| tacattgggt caatgggagg tgagccaatg ggaaaaaccc attgctgcca agtacactga | 420 |
| ctcaataggg actttccaat gggtttttcc attgttggca agcatataag gtcaatgtgg | 480 |
| gtgagtcaat agggactttc cattgtattc tgcccagtac ataaggtcaa tagggggtga | 540 |
| atcaacagga aagtcccatt ggagccaagt acactgcgtc aatagggact ttccattggg | 600 |
| ttttgcccag tacataaggt caatagggga tgagtcaatg ggaaaaaccc attggagcca | 660 |
| agtacactga ctcaataggg actttccatt gggttttgcc cagtacataa ggtcaatagg | 720 |
| gggtgagtca acaggaaagt cccattggag ccaagtacat tgagtcaata gggactttcc | 780 |
| aatgggtttt gcccagtaca taaggtcaat gggaggtaag ccaatgggtt tttcccatta | 840 |
| ctggcacgta tactgagtca ttagggactt tccaatgggt tttgcccagt acataaggtc | 900 |
| aataggggtg aatcaacagg aaagtcccat tggagccaag tacactgagt caatagggac | 960 |
| tttccattgg gttttgccca gtacaaaagg tcaatagggg gtgagtcaat gggtttttcc | 1020 |
| cattattggc acgtacataa ggtcaatagg ggtgagtcat tgggtttttc cagccaattt | 1080 |
| aattaaaacg ccatgtactt tcccaccatt gacgtcaatg ggctattgaa actaatgcaa | 1140 |
| cgtgaccttt aaacggtact ttcccatagc tgattaatgg gaaagtaccg ttctcgagcc | 1200 |
| aatacacgtc aatgggaagt gaaagggcag ccaaaacgta acaccgcccc ggttttcccc | 1260 |
| tggaaattcc atattggcac gcattctatt ggctgagctg cgttctacgt gggtataaga | 1320 |
| ggcgcgacca gcgtcggtac cgtcgcagtc ttcggtctga ccaccgtaga acgcagagct | 1380 |
| cctcgctgca ggcggccgct ctagaactcg tcgatcgcag cgatgacaaa cctgcaagat | 1440 |
| caaacccaac agattgttcc gttcatacgg agccttctga tgccaacaac cggaccggcg | 1500 |
| tccattccgg acgacaccct ggagaagcac actctcaggt cagagacctc gacctacaat | 1560 |
| ttgactgtgg gggacacagg gtcagggcta attgtctttt tccctggatt ccctggctca | 1620 |
| attgtgggtg ctcactacac actgcagagc aatgggaact acaagttcga tcagatgctc | 1680 |
| ctgactgccc agaacctacc ggccagctac aactactgca gactagtgag tcggagtctc | 1740 |
| acagtgaggt caagcacact ccctggtggc gtttatgcac taaacggcac cataaacgcc | 1800 |
| gtgaccttcc aaggaagcct gagtgaactg acagatgtta gctacaatgg gttgatgtct | 1860 |
| gcaacagcca acatcaacga caaaattggg aatgtcctgg taggggaagg ggtcactgtc | 1920 |
| ctcagcctac ccacatcata tgatcttggg tatgtgaggc ttggtgaccc cattcccgct | 1980 |
| atagggcttg acccaaaaat ggtagctaca tgcgacagca gtgacaggcc cagagtctac | 2040 |
| accataactg cagccgatga ttaccaattc tcatcacagt accaaccagg tggggtaaca | 2100 |
| atcacactgt tctcagccaa cattgatgct atcacaagcc tcagcattgg gggagagctc | 2160 |
| gtgtttcaaa caagcgtcca aggccttgta ctgggcgcca ccatctacct tataggcttt | 2220 |
| gatgggactg cggtaatcac cagagctgta gccgcagata atgggctgac ggccggcacc | 2280 |
| gacaatctta tgccattcaa tcttgtcatt ccaaccaatg agataaccca gccaatcaca | 2340 |
| tccatcaaac tggagatagt gacctccaaa agtggtggtc aggcagggga tcagatgtca | 2400 |
| tggtcggcaa gtgggagcct agcagtgacg atccatggtg gcaactatcc aggggccctc | 2460 |
| cgtcccgtca cactagtagc ctacgaaaga gtggcaacag gatccgtcgt tacggtcgct | 2520 |
| ggggtgagta acttcgagct gattccaaat cctgaactag caaagaacct ggttacagaa | 2580 |
| tacggccgat ttgacccagg agccatgaac tacacaaaat tgatactgag tgagagggac | 2640 |
| cgtcttggca tcaagaccgt ctggccaaca agggagtaca ctgattttcg tgagtacttc | 2700 |
| atggaggtgg ccgacctcaa ctctcccctg aagattgcag gagcatttgg cttcaaagac | 2760 |
| ataatccggg ctataaggag gtaagcttga tctagagcgg ccgcggggat ccagacatga | 2820 |
| taagatacat tgatgagttt ggacaaacca caactagaat gcagtgaaaa aaatgcttta | 2880 |
| tttgtgaaat ttgtgatgct attgctttat ttgtaaccat tataagctgc aataaacaag | 2940 |
| ttaacaacaa caattgcatt cattttatgt ttcaggttca gggggaggtg tgggaggttt | 3000 |
| tttcggatcc tctagagtcg acaattattt catttaataa catatagccc aaagacctct | 3060 |
| atgaacattt agtttcccgt atactcaacg gcgcgtgtac acacaagggc gaattccaca | 3120 |
| gtggatatca agcttaatta agtaccgagc tcgaattggc gcgccaggtc aattccctgg | 3180 |
| cattatgccc agtacatgac cttatgggac tttcctactt ggcagtacat ctacgtatta | 3240 |
| gtcatcgcta ttaccatggt gatgcggttt tggcagtaca tcaatgggcg tggatagcgg | 3300 |
| tttgactcac ggggatttcc aagtctccac cccattgacg tcaatgggag tttgttttgg | 3360 |
| caccaaaatc aacgggactt tccaaaatgt cgtaacaact ccgccccatt gacgcaaatg | 3420 |
| ggcggtaggc gtgtacggtg ggaggtctat ataagcagag ctcgtttagt gaaccgtcag | 3480 |
| atcgcctgga gacgccatcc acgctgtttt gacctccata gaagacaccg ggcgcgccgg | 3540 |
| atccatgggc cccagacctt ctaccaagaa cccagtacct atgatgctga ctgtccgagt | 3600 |
| cgcgctggta ctgagttgca tctgtccggc aaactccatt gatggcaggc ctcttgcggc | 3660 |
| tgcaggaatt gtggttacag gagacaaagc cgtcaacata tacacctcat cccagacagg | 3720 |
| atcaatcata gttaagctcc tcccgaatct gcccaaggat aaggaggcat gtgcgaaagc | 3780 |
| ccccttggat gcatacaaca ggacattgac cactttgctc accccccttg gtgactctat | 3840 |
| ccgtaggata caagagtctg tgactacatc tggagggggg agacaggggc gccttatagg | 3900 |
| cgccattatt ggcggtgtgg ctcttggggt tgcaactgcc gcacaaataa cagcggccgc | 3960 |
| agctctgata caagccaaac aaaatgctgc caacatcctc cgacttaaag agagcattgc | 4020 |
| cgcaaccaat gaggctgtgc atgaggtcac tgacggatta tcgcaactag cagtggcagt | 4080 |
| tgggaagatg cagcagtttg ttaatgacca atttaataaa acagctcagg aattagactg | 4140 |
| catcaaaatt gcacagcaag ttggtgtaga gctcaacctg tacctaaccg aattgactac | 4200 |
| agtattcgga ccacaaatca cttcacctgc tttaaacaag ctgactattc aggcacttta | 4260 |
| caatctagct ggtggaaata tggattactt attgactaag ttaggtgtag ggaacaatca | 4320 |
| actcagctca ttaatcggta gcggcttaat caccggtaac cctattctat acgactcaca | 4380 |
| gactcaactc ttgggtatac aggtaactct accttcagtc gggaacctaa ataatatgcg | 4440 |
| tgccacctac ttggaaacct tatccgtaag cacaaccagg ggatttgcct cggcacttgt | 4500 |
| cccaaaagtg gtgacacagg tcggttctgt gatagaagaa cttgacacct catactgtat | 4560 |
| agaaactgac ttagatttat attgtacaag aatagtaacg ttccctatgt cccctggtat | 4620 |
| ttattcctgc ttgagcggca atacgtcggc ctgtatgtac tcaaagaccg aaggcgcact | 4680 |
| tactacacca tacatgacta tcaaaggttc agtcatcgcc aactgcaaga tgacaacatg | 4740 |
| tagatgtgta aaccccccgg gtatcatatc gcaaaactat ggagaagccg tgtctctaat | 4800 |
| agataaacaa tcatgcaatg ttttatcctt aggcgggata actttaaggc tcagtgggga | 4860 |
| attcgatgta acttatcaga agaatatctc aatacaagat tctcaagtaa taataacagg | 4920 |
| caatcttgat atctcaactg agcttgggaa tgtcaacaac tcgatcagta atgctttgaa | 4980 |
| taagttagag gaaagcaaca gaaaactaga caaagtcaat gtcaaactga ctagcacatc | 5040 |
| tgctctcatt acctatatcg ttttgactat catatctctt gtttttggta tacttagccc | 5100 |
| gattctagca tgctacctaa tgtacaagca aaaggcgcaa caaaagacct tattatggct | 5160 |
| tgggaataat actctagatc agatgagagc cactacaaaa atgtgaggat ctctcgagga | 5220 |
| attctagatc ccacgtcact attgtatact ctatattata ctctatgtta tactctgtaa | 5280 |
| tcctactcaa taaacgtgtc acgcctgtga aaccgtacta agtctcccgt gtcttcttat | 5340 |
| caccatcagg tgacatcctc gcccaggctg tcaatcatgc cggtatcgat tccagtagca | 5400 |
| ccggccccac gctgacaacc cactcttgca gcgttagcag cgcccctctt aacaagccga | 5460 |
| cccccaccag cgtcgcggtt actaacactc ctctcccc | 5498 |

| | |
|---|---|
| <210> | 2 |
| <211> | 3157 |
| <212> | DNA |
| <213> | Artificial Sequence |
| <220> | |
| <223> | Expression cassette with ILTV gD and gI genese |
| <400> | 2 |

| | |
|---|---|
| aagcttaatt aacgtcgacg gcagagtcgc agacgcccct attggacgtc aaaattgtag | 60 |
| aggtgaagtt ttcaaacgat ggcgaagtaa cggcgacttg cgtttccacc gtcaaatctc | 120 |
| cctatagggt agaaactaat tggaaagtag acctcgtaga tgtaatggat gaaatttctg | 180 |
| ggaacagtcc cgccggggtt tttaacagta atgagaaatg gcagaaacag ctgtactaca | 240 |
| gagtaaccga tggaagaaca tcggtccagc taatgtgcct gtcgtgcacg agccattctc | 300 |
| cggaacctta ctgtcttttc gacacgtctc ttatagcgag ggaaaaagat atcgcgccag | 360 |
| agttatactt tacctctgat ccgcaaacgg catactgcac aataactctg ccgtccggcg | 420 |
| ttgttccgag attcgaatgg agccttaata atgtttcact gccggaatat ttgacggcca | 480 |
| cgaccgttgt ttcgcatacc gctggccaaa gtacagtgtg gaagagcagc gcgagagcag | 540 |
| gcgaggcgtg gatttctggc cggggaggca atatatacga atgcaccgtc ctcatctcag | 600 |
| acggcactcg cgttactacg cgaaaggaga ggtgcttaac aaacacatgg attgcggtgg | 660 |
| aaaacggtgc tgctcaggcg cagctgtatt cactcttttc tggacttgtg tcaggattat | 720 |
| gcgggagcat atctgctttg tacgcaacgc tatggaccgc catttatttt tgaggaatgc | 780 |
| tttttggact atcgtactgc tttcttcctt cgctagccag agcaccgccg ccgtcacgta | 840 |
| cgactacatt ttaggccgtc gcgcgctcga cgcgctaacc ataccggcgg ttggcccgta | 900 |
| taacagatac ctcactaggg tatcaagagg ctgcgacgtt gtcgagctca acccgatttc | 960 |
| taacgtggac gacatgatat cggcggccaa agaaaaagag aaggggggcc ctttcgaggc | 1020 |
| ctccgtcgtc tggttctacg tgattaaggg cgacgacggc gaggacaagt actgtccaat | 1080 |
| ctatagaaaa gagtacaggg aatgtggcga cgtacaactg ctatctgaat gcgccgttca | 1140 |
| atctgcacag atgtgggcag tggactatgt tcctagcacc cttgtatcgc gaaatggcgc | 1200 |
| gggactgact atattctccc ccactgctgc gctctctggc caatacttgc tgaccctgaa | 1260 |
| aatcgggaga tttgcgcaaa cagctctcgt aactctagaa gttaacgatc gctgtttaaa | 1320 |
| gatcgggtcg cagcttaact ttttaccgtc gaaatgctgg acaacagaac agtatcagac | 1380 |
| tggatttcaa ggcgaacacc tttatccgat cgcagacacc aatacacgac acgcggacga | 1440 |
| cgtatatcgg ggatacgaag atattctgca gcgctggaat aatttgctga ggaaaaagaa | 1500 |
| tcctagcgcg ccagaccctc gtccagatag cgtcccgcaa gaaattcccg ctgtaaccaa | 1560 |
| gaaagcggaa gggcgcaccc cggacgcaga aagcagcgaa aagaaggccc ctccagaaga | 1620 |
| ctcggaggac gacatgcagg cagaggcttc tggagaaaat cctgccgccc tccccgaaga | 1680 |
| cgacgaagtc cccgaggaca ccgagcacga tgatccaaac tcggatcctg actattacaa | 1740 |
| tgacatgccc gccgtgatcc cggtggagga gactactaaa agttctaatg ccgtctccat | 1800 |
| gcccatattc gcggcgttcg tagcctgcgc ggtcgcgctc gtggggctac tggtttggag | 1860 |
| catcgtaaaa tgcgcgcgta gctaatcgag cctagaatag gtggtttctt cctacatgcc | 1920 |
| acgcctcacg ctcataatat aaatcacatg gaatagcata ccaatgccta ttcattggga | 1980 |
| cgttcgaaaa gcatggcatc gctacttgga actctggctc tccttgccgc gacgctcgca | 2040 |
| cccttcggcg cgatgggaat cgtgatcact ggaaatcacg tctccgccag gattgacgac | 2100 |
| gatcacatcg tgatcgtcgc gcctcgcccc gaagctacaa ttcaactgca gctatttttc | 2160 |
| atgcctggcc agagacccca caaaccctac tcaggaaccg tccgcgtcgc gtttcggtct | 2220 |
| gatataacaa accagtgcta ccaggaactt agcgaggagc gctttgaaaa ttgcactcat | 2280 |
| cgatcgtctt ctgtttttgt cggctgtaaa gtgaccgagt acacgttctc cgcctcgaac | 2340 |
| agactaaccg gacctccaca cccgtttaag ctcactatac gaaatcctcg tccgaacgac | 2400 |
| agcgggatgt tctacgtaat tgttcggcta gacgacacca aagaacccat tgacgtcttc | 2460 |
| gcgatccaac tatcggtgta tcaattcgcg aacaccgccg cgactcgcgg actctattcc | 2520 |
| aaggcttcgt gtcgcacctt cggattacct accgtccaac ttgaggccta tctcaggacc | 2580 |
| gaggaaagtt ggcgcaactg gcaagcgtac gttgccacgg aggccacgac gaccagcgcc | 2640 |
| gaggcgacaa ccccgacgcc cgtcactgca accagcgcct ccgaacttga agcggaacac | 2700 |
| tttacctttc cctggctaga aaatggcgtg gatcattacg aaccgacacc cgcaaacgaa | 2760 |
| aattcaaacg ttactgtccg tctcgggaca atgagcccta cgctaattgg ggtaaccgtg | 2820 |
| gctgccgtcg tgagcgcaac gatcggcctc gtcattgtaa tttccatcgt caccagaaac | 2880 |
| atgtgcaccc cgcaccgaaa attagacacg gtctcgcaag acgacgaaga acgttcccaa | 2940 |
| actagaaggg aatcgcgaaa atttggaccc atggttgcgt gcgaaataaa caagggggct | 3000 |
| gaccaggata gtgaacttgt ggaactggtt gcgattgtta acccgtctgc gctaagctcg | 3060 |
| cccgactcaa taaaaatgtg aagatctctt aattaacaat aaacatagca tacgttatga | 3120 |
| catggtctac cgcgtcttat atggggacga caagctt | 3157 |

| | |
|---|---|
| <210> | 3 |
| <211> | 20 |
| <212> | DNA |
| <213> | Artificial Sequence |
| <220> | 3 |
| <223> | guide RNA for insertion between UL44 and UL45 genes of HVT |
| <400> | |

| | |
|---|---|
| acatcgggac gtacatcatg | 20 |

| | |
|---|---|
| <210> | 4 |
| <211> | 20 |
| <212> | DNA |
| <213> | Artificial Sequence |
| <220> | |
| <223> | guide RNA for insertion between UL45 and UL46 genes of HVT |
| <400> | 4 |

| | |
|---|---|
| ctaacggtta ctgtgtttta | 20 |

| | |
|---|---|
| <210> | 5 |
| <211> | 20 |
| <212> | DNA |
| <213> | Artificial Sequence |
| <220> | |
| <223> | guide RNA for insertion between UL54 and LORF3 genes of HVT |
| <400> | 5 |

| | |
|---|---|
| ttagatttcc ggacagcctg | 20 |

| | |
|---|---|
| <210> | 6 |
| <211> | 20 |
| <212> | DNA |
| <213> | Artificial Sequence |
| <220> | |
| <223> | guide RNA for insertion into LORF3 of HVT |
| <400> | 6 |

| | |
|---|---|
| taccacgccg aggcgcctat | 20 |

### EXAMPLES

### Example 1: Construction and In Vitro Testing of Multivalent rHVT Vectors

### 1.1. Constructs Made and Tested

Based on the HVT vector construct rHVT-VP2-F (HVP360; WO 2016/102647), a series of HVT recombinants were made that additionally expressed the ILTV gD and gI genes from the vector's UL genome region. HVP360 expresses the IBDV-VP2 and NDV-F genes from one expression cassette, that is inserted in the HVT Us2 gene. Using the CRISPR/Cas9 technique as described by Tang et al. 2018 (supra), a further cassette expressing ILTV gD-gI was introduced into the UL region of the HVP360 genome, at one of different sites. Several constructs were made by insertion of the second expression cassette, and one rHVT-VP2-F-gD-gI construct (the rHVT according to the present invention) was found to have excellent levels of stable replication, heterologous gene expression, and vaccination efficacy, when tested in vitro and in vivo.

Some of the rHVT-VP2-F vector constructs with additional insert of the gD-gI expression cassette are described here, to allow comparisons, and illustrate the advantageous effects of the present invention. The insertion site nucleotide numbers are indicated relative to the genome of HVT strain FC-126 as published in GenBank accession nr. AF291866:
rHVT construct HVP412: having the gD-gI cassette inserted between UL44 and UL45, specifically: between nt. 94482 and 94483,
rHVT construct HVP413: having the gD-gI cassette inserted between UL45 and UL46, specifically: between nt. 95335 and 95336,
rHVT construct G7 (the rHVT according to the present invention): having the gD-gI cassette inserted between UL54 and LORF3, specifically: between nt. 110.395 and 110.396, and
rHVT construct G8: having the gD-gI cassette inserted in the LORF3 gene ORF, specifically: between nt. 111.219 and 111.220.

Constructs HVP412, HVP413, and G8 are included here as comparative examples; constructs HVP412 and HVP413 have been described in more detail in PCT/EP2021/087445.

The guide RNA sequences used for the CRISPR/Cas9-directed insertions of the gD-gI cassette in HVT UL are:

-insertion between UL44 and UL45:
(SEQ ID NO: 3)
5′-ACATCGGGACGTACATCATG-3′,
-insertion between UL45 and UL46:
(SEQ ID NO: 4)
5′-CTAACGGTTACTGTGTTTTA-3′,
-insertion between UL54 and LORF3:
(SEQ ID NO: 5)
5′-TTAGATTTCCGGACAGCCTG-3′,
-insertion in LORF3:
(SEQ ID NO: 6)
5′-TACCACGCCGAGGCGCCTAT-3′.

The SEQ ID NOs 3-6 are indicated here in DNA code, as they were inserted into a DNA plasmid, and were then transcribed to produce the guide RNA's, all using standard procedures. With the guide RNA of SEQ ID NO: 4, the cut is made between its nucleotides 3 and 4; with the guide RNA of SEQ ID NOS: 3, 5 and 6, the cut is made between their nucleotides 17 and 18.

The guide RNA's were designed using the Internet website: zlab.bio/guide-design-resources. One of many alternatives, is to use the program Geneious Prime® (Biomatters Ltd., New Zealand).

A graphic representation of the expression cassettes used, and their insertion into the HVT genome for the rHVT construct G7 of the present invention is given in FIG. 1.

Other insertions of the gD-gI expression cassette into the UL region of the vector construct rHVT-VP2-F had been constructed and were tested previously, by insertion:
intragenic in UL39-central,
intragenic in UL39-near the 3′ end,
intergenic between UL40 and UL41, and
intergenic between UL47 and UL48.

### 1.2. Genetic Stability In Vitro

The various rHVT-VP2-F-gD-gI constructs, were passaged on CEF cells in vitro for 15 consecutive rounds. The P15 plaques were monitored for the expression of the inserted genes by IFA as follows: overnight established CEF monolayers were infected with one of the rHVT vectors at 15^{th} passage level. Plates were incubated for 2-3 days until CPE was clearly visible, and then fixated with 96% ethanol. Expression of VP2 and F were detected with specific monoclonal antibodies; gD and gI were detected using chicken polyclonal anti-ILTV antibodies. After the first antibody an Alexa™ labelled conjugate was used as secondary antibody. Next, plates were read by UV microscopy. About 100 plaques were counted for each of the recombinants to assess expression.

For the different rHVT constructs with F and VP2 genes in Us2, and ILTV gD-gI genes in a locus in the UL region, it was observed that several times plaques were lost in a later passage, or that plaques appeared which no longer showed expression of one or more of the heterologous gene-inserts. Nevertheless, for all rHVT-VP2-F-gD-gI constructs at least one clone could be isolated that could replicate well in vitro, and that maintained stable expression of the heterologous inserts, for 15 passages in cell culture.

Next these rHVT-VP2-F-gD-gI isolates were tested for their replication and expression in vivo.

### Example 2: Characterisation of Multivalent rHVT Vectors In Vivo

### 2.1. Introduction

In several in vivo experiments the viral replication, the expression of gene-inserts, and the induction of a serological immune response, was tested of the various multivalent rHVT vectors that were constructed as described in Example 1. Experimental animals were SPF layer chickens, of 1-day old. To determine replication in vivo of the rHVT vector vaccines, HVT viremia levels at day 15, 21 or 25 post vaccination were determined. To check for expression of the heterologous gene inserts and induction of specific antibodies, blood samples were taken from the inoculated chickens at different time points during the trials.

### 2.2. Experimental

Group size was 10-15 animals, plus 5 hatchmates. Blood samples taken from the hatch mates at day of vaccination were serologically tested to assure the batch of animals was negative for antibodies against NDV, IBDV and ILTV on the day of vaccination.

rHVT vaccine viruses were used at 15^{th} or 16^{th} cell-passage level, and were stored as infected CEF in liquid nitrogen. Viral titres (in infected cells) of the vaccine stocks were 0.4-1.2×10\hat{ }6 pfu/ml. Vaccine dose was 2000 PFU per animal in 0.2 ml of standard HVT/CEF diluent, which was inoculated subcutaneously in the neck, using standard procedures.

Control groups were either unvaccinated, or received the rHVT parent vector HVP360 as vaccine. No acclimatization was needed as the chicks were placed into negative pressure isolators shortly after hatch, and were labelled and vaccinated shortly thereafter.

Blood samples were taken from the vaccinated chicks on convenient days post vaccination, e.g. at 15 or 25 days p.v. In the various vaccination-challenge trials (described below) samples for serology were taken shortly before the challenge, thus at 3, 4, or 5 weeks p.v. Blood samples were collected from the wing vein into tubes with clot activator, and kept at ambient temperature until processing.

### 2.2.1. Viremia

Viremia sampling from spleen was done as follows: at day 15 or 21 p.v., spleens were isolated post-mortem from 5-6 chicks per group. Clean tweezers were used for each chick. Spleens were collected in tubes with 5 ml of 10 mM PBS with phenol red indicator and antibiotics, and kept on ice until processing. Next, spleens were homogenised, taken up into fresh medium and cells were counted. To determine rHVT viremia per 5.0×10\hat{ }6 spleen cells, that number of cells was added to a dish with an established CEF monolayer, and incubated at 38° C. for 3-4 days.

Alternatively, viremia was determined from peripheral blood lymphocytes (PBL) isolated from blood samples taken from the vaccinated chicks at 3 or 4 weeks p.v. Then 5×10\hat{ }6 PBL were added to CEF monolayers and incubated.

If rHVT virus was present in the cells, it caused a cytopathogenic effect on the CEFs which was visible as plaques. 3 plates were counted per animal sample. Plates were fixated using 96% ethanol and an immunofluorescence assay (IFA) was applied to stain the virus-infected cells with anti-HVT antisera in combination with staining for one of the antigens VP2, F, or gD-gI. Consequently, three separate double stainings were performed.

### 2.2.2. Serology

Blood samples for testing of serological responses were taken at 2-5 weeks p.v. The samples were centrifuged, serum was collected, and complement was inactivated. The sera were used in different tests to determine the seroresponse of the vaccinated chickens against the expressed heterologous genes: IBDV-VP2 response was measured by virus-neutralisation (VN) assay using classic IBDV virus strain D78; serological responses against NDV-F, IBDV-VP2, and ILTV-gD-gI were measured by ELISA and expressed in units relative to the results of standard samples.

### 2.3. Results and Conclusions

### 2.3.1. Viremia

rHVT viremia was detected at 15 days p.v. in spleens from 6 animals per group, or in PBL at 25 days p.v. from 5 animals/group.

The average spleen viremia numbers per group receiving one of the rHVT vaccines was for:
the control vector vaccine HVP360: 93 PFU/5 million spleen cells,
the rHVT with gD-gI insert in UL39-centre, or inserted near the 3′ end of the UL39 gene: HVT viremia was undetectable; no replication in vivo,
the rHVT with gD-gI insert in UL40-41 or in UL47-48, scored: 36, respectively 45 PFU/5×10\hat{ }6. This means a viremia lower than that of HVP360 control, and thus a reduced replication in vivo.

The average viremia per group measured in PBLs, and expressed in PFU/5×10\hat{ }6 PBL was for:
HVP360: 17,
HVP412: 15,
HVP413: 9,
G7: 19, and for
G8: 8.

In conclusion: all rHVT vector constructs HVP412, HVP413, and G7 had good viremia after vaccination, comparable to that of HVP360; viraemia of the G8 constructs was moderate. However, the viremia for the rHVT with the gD-gI insert between UL54 and LORF3 (the rHVT according to the present invention), was the best of the tested group.

### 2.3.2. Genetic Stability In Vivo

The rHVT viruses obtained from vaccinated chickens were tested for continued expression of the heterologous gene inserts. From all isolates from spleen at 15 days p.v., about 100 rHVT plaques were analysed by IFA.

Both rHVT vectors with gD-gI insert in UL39 did not replicate in vivo, therefore no stability could be determined.

The rHVT vector with gD-gI insert in UL40-41 demonstrated genetic instability after replication in vivo for 15 days, as only 75% of the plaques tested showed expression of the NDV-F gene, and only half of the plaques showed expression of the IBDV-VP2 gene. Similarly, the rHVT with gD-gI between UL47 and UL48 showed plaques that had lost the expression of the gD and gI genes.

For the other rHVT constructs: rHVT with gD-gI inserts in UL44-45 (HVP412), in UL45-46 (HVP413), in UL54-LORF3 (G7), or inside LORF3 (G8), all plaques maintained the expression of all the heterologous genes: IBDV-VP2, NDV-F, and ILTV-gD and gI, after replication in vivo for 15 days. Of these, the G7 construct (the rHVT according to the present invention) showed full expression of all heterologous inserts even at 21 days p.v.

### 2.3.3. Serology

In nature, the immuneresponse against the pathogens from which the three heterologous antigens were derived: IBDV, NDV, and ILTV, all rely to a large extent on a humoral immune response. Consequently, a measurement of the antibody response generated by vaccination, is a reliable correlate of in vivo protection against infection and/or disease from these pathogens.

The serological responses induced by the vaccination of chickens with the various rHVT vectors were analysed by ELISA. Negative controls were the hatchmates from the same batch of animals, that were tested before the vaccinations. Positive control for the seroprotection against NDV and IBDV was the vaccination with the HVP360 vector.

None of the hatchmates tested at day 1 had any detectable antibody titres against one of the pathogens NDV, IBDV, or ILTV.

The anti-ILTV seroresponse was tested using a commercial ELISA test (ID Screen™ ILT gI Indirect, from ID vet). In this test, result values above 611 indicate a protective immune-response.

Results of the average ELISA scores (n=6) for the different groups at specific days after vaccination, are presented in Table 3.

TABLE 3
ELISA scores for anti-ILTV seroresponse,
induced by vaccination with rHVT vectors.

avg. anti-ILTV ELISA titre at day X p.v.

| gD-gI insertion | D 22 | D 32 | D 42 |
|---|---|---|---|
| UL44-45 | 207 | 867 | 709 |
| UL45-46 | 67 | 818 | 721 |
| UL47-48 | 6 | 60 | 123 |

As is clear from the ELISA results presented in Table 3, the rHVT with gD-gI inserted between UL47-48 induced only very low levels of anti ILT antisera, at each of the days 22, 32, and 42 p.v. tested; thus always below protective levels. Consequently, the rHVT with gD-gI insert between UL47-48, even though stable in vitro and in vivo, would not be useful as a multivalent vector vaccine against ILTV infection.

However the rHVT constructs with gD-gI inserted between UL44-45 (HVP412) or between UL45-46 (HVP413) scored well above protective levels at days 32 and 42 p.v.

In addition, the seroresponses against NDV and IBDV induced by the vaccination with constructs HVP412 and HVP413 showed ELISA score values very close to those induced by the parental vector HVP360, at all days tested. Consequently, in these constructs HVP412 and HVP413, the expression and delivery of the NDV-F and IBDV-VP2 genes had not been affected by the additional insertion of the ILTV gD and gI genes into the UL region.

### 2.3.4. Further Serology Data from Comparative Experiments with rHVT-VP2-F-gD-gI Constructs

As described above, similar serology tests were done on samples obtained from chicks vaccinated with one of the constructs: HVP412, HVP413, G7 and G8, in the context of several comparative vaccination-challenge experiments that are described in detail below.

### IBDV Serology

The IBDV serology (measured by ID VET ID Screen® IBD VP2 Elisa) induced by the 4 rHVT-VP2-F-gD-gI constructs was slightly below that induced by HVP360 at 3 weeks p.v., but reached the levels of HVP360 at 4 weeks p.v. The rHVT-VP2-F-gD-gI constructs induced average anti-IBDV titres between 11 and 12 Log2 at 4 weeks p.v., with the titres for HVP412 and G8 below those of HVP413 and G7. The protective titre is at about 10.2 Log2.

### NDV Serology

Like against IBDV, the NDV serological response (measured by ID VET ID Screen® ND Elisa) induced by the 4 rHVT-VP2-F-gD-gI constructs tested, was slightly delayed as compared to that induced by the rHVT-VP2-F (HVP360) construct itself: while HVP360 already showed an average anti-NDV titre of 10.4 Log2 at 3 weeks p.v., the 4 rHVT-VP2-F-gD-gI constructs only showed titres between 7 and 9 Log 2 at that time. However this quickly picked up, and at 4 weeks p.v. all 4 rHVT-VP2-F-gD-gI constructs induced good anti NDV titres of around 11 Log 2, near to that for HVP360, whereby the titres from G7 and G8 vaccination were slightly below those induced by HVP412 and HVP413. The protective titre is at about 9.7 Log2.

### ILTV Serology

The ILTV serology (measured by ID VET ID Screen® ILTV Elisa) induced by the 4 rHVT-VP2-F-gD-gI constructs showed a distinct division, both at 3 and at 4 weeks p.v.: the anti-ILTV titres induced by the HVP412 and the HVP413 construct were 1.5-2 Log points below those induced by the G7 and G8 constructs, which were at 9.1 and 9.2 Log2 at 3 weeks, and at 10.6 and 10.5 Log2 at 4 weeks p.v., respectively. The fact that most titres were below the alleged protective titre indicated by the test manufacturer, at 9.2 Log2, did not correspond to the excellent clinical protection observed in this experiment. Consequently, for this test, serology did not correlate well with in vivo protection.

### Example 3: Vaccination-Challenge Trials

### 3.1. Introduction

To expand on the in vivo serology data described in Example 2, several vaccination-challenge trials were done, in which we compared side-by-side the vaccine efficacy of the rHVT-VP2-F-gD-gI constructs that were found to be stable in vivo: HVP412, HVP413, G7 and G8. These trials were all performed in SPF chickens that were vaccinated at day-old, and were given a challenge infection at different times p.v. with a virulent strain of one of the pathogens NDV, IBDV, or ILTV. During these experiments several samples were taken and parameters of infection and vaccine efficacy were measured. These challenges were done largely as described before, e.g. the NDV- or IBDV challenges as described in WO 2016/102647, and the ILTV challenge as described in WO 2019/072964.

### 3.2. Materials and Methods

### 3.2.1. Common to the Vaccination Trials

SPF White leghorn layer chickens were hatched in isolators; each chick was individually marked with tag-numbers, and were vaccinated the same day by subcutaneous route in the neck, with 2000 pfu (=10\hat{ }3.3) of one of the rHVT-VP2-F-gD-gI constructs: HVP412, HVP413, G7 or G8, at cell passage level 15 or 16, that was administered as infected CEFs, at 0.2 ml/dose in standard diluent.
as positive control was used a similar dose of one of the commercial bivalent rHVT vector vaccines: either the HVT-ND-IBD (HVP360, WO 2016/102647; Innovax® ND-IBD {MSD Animal Health}) vector, or the HVT-ND-ILT vector disclosed in WO 2013/057236 (Innovax® ND-ILT {MSD Animal Health}).
negative controls were unvaccinated, and their group size was 9 or 10 chicks/group.
for the various vaccination-challenge trials group size was 10, 11, or 15 chicks/group; each group was housed in a different isolator. For each experiment 5 or 10 hatchmates were bled for confirming seronegative status at the start of the experiments.
inoculation, viremia, and expression was verified by testing spleen- or PBL samples at 2 or 3 weeks p.v., as described in Example 2.

### 3.2.2. Details of the Challenges Applied

### IBDV Challenge

IBDV challenges were done according to European Pharmacopoeia (Ph. Eur.) monograph 0587, with a group size of 10 chicks. At 3 weeks p.v., each chick received 30 chicken infective dose 50% (CID50) of IBDV strain CS89, by eyedrop, in 0.1 ml PBS, divided over both eyes.

After challenge the chicks were monitored during 10 days for clinical signs of IBD, and a clinical score was assigned to each chick daily on a scale from 0-3 for: no signs—some signs—severe signs—death, respectively. Clinical signs of IBD are: depression, huddling, paleness, anorexia, ruffled feathers, and abnormal faeces. Next, all remaining birds were euthanised and the bursae were scored macroscopically, and sampled for histological analysis, using common criteria such as: macroscopically: enlargement and oedema; and microscopically: the percentage of follicles that showed lymphoid depletion, necrosis, and influx of heterophils (in blocks of 25%). An observation in any of these tests also contributed to the total clinical score.

### NDV Challenge

The NDV challenge was done according to Ph. Eur. monograph 0450, using 15 chicks/group. Specifically: the challenge was given at 5 weeks p.v., by administering per chick 5 Log10 EID50 of NDV strain Herts 33/56, in 0.2 ml PBS, given by intramuscular route. A group of 9 unvaccinated chicks served as challenge controls. Chicks were observed daily for maximally 14 days post challenge, and an NDV clinical score was assigned to each chick daily on a scale from 0-3 for: no signs—some signs—severe signs—death, respectively. Typical signs of NDV infection are neurological symptoms, e.g.: twisted neck, wing clearly hanging down, uncoordinated walk, paralysis, muscle tremor, and body curled backwards. At day 21 p.v. (the day before challenge) blood samples were taken from 5 chicks/group to confirm viremia in PBLs, as well as serology and heterologous gene-expression.

### ILTV Challenge

The ILTV challenges were done according to Ph. Eur. monograph 1068, starting with 11 chicks per group. The challenge was administered at 4 weeks p.v., to 10 chicks per group, receiving 3.0 Log10 EID50 of ILTV strain 96-3, as a CAM homogenate in standard cell-culture medium. The ILTV challenge virus was administered intra-tracheally, via syringe to the middle part of the trachea, at 0.1 ml/chick.

For 7 days post challenge the chicks were observed twice daily (mornings and evenings) for signs of ILT; each morning a clinical score was assigned to each chick on a scale from 0-3 for: no signs—some signs—severe signs—death, respectively. Clinical signs typical for ILTV infection are: marked dyspnoea, laboured breathing, gasping, expectoration of blood, nasal discharge, conjunctivitis, and swelling of the sinuses.

At 7 days after challenge, the chicks were euthanised and tracheas were isolated and scored for signs of ILTV infection, by checking for: redness and type- and consistency of content. An observation in any of these tests also contributed to the total clinical score.

At 28 days p.v. (the day before challenge) blood samples were taken from 10 chicks/group to confirm viremia, serology, and heterologous gene-expression.

### 3.3. Results

### Common Findings from the Challenges

All vaccinates receiving the HVP412, HVP413, G7 or G8 vaccine showed good viremia of the rHVT vector, and a good expression of all the heterologous gene inserts at 3, 4 or 5 weeks post vaccination, as described in Example 2.

For all the comparative challenge trials, the hatchmates tested at day 1 were seronegative for antibodies against NDV, IBDV, and ILTV.

The total clinical scores used for the ILTV and IBDV results, are the summation of all clinical scores assigned over the observation period to all of the 10 chicks per group, as well as scores resulting from the macroscopic- and microscopic examinations done.

### IBDV Challenge Infection

IBDV vaccination efficacy was tested at 3 weeks p.v., and all of the four rHVT-VP2-F-gD-gI constructs tested demonstrated excellent IBDV vaccine efficacy, as all the chicks in these groups were 100% protected. This even though the challenge was severe: all the 10 negative control chicks showed severe signs of IBDV infection on day 3 or 4 post challenge, and all died or had to be euthanised.

Within the protected groups a slight differentiation could be made based on the very modest residual clinical signs that were observed: the HVP412 vaccine group had a total clinical score of 7; the HVP413 group had 5; and the G7 group only had a total clinical score 2.

Consequently, all rHVT-VP2-F-gD-gI constructs induced excellent protection against IBDV challenge infection, but the vector vaccine of the G7 construct (the rHVT according to the invention) resulted in almost no clinical signs anymore, even less than those in the HVP412- and HVP413 test groups.

### NDV Challenge Infection

The protection induced by the multivalent rHVT-VP2-F-gD-gI vector vaccines against a severe NDV challenge infection was tested at 5 weeks post vaccination. The severity of the challenge infection was confirmed in that all of the non-vaccinated controls showed extensive clinical signs of NDV infection within 48 hours, and all had to be euthanised for humane reasons.

The HVP412 construct could only protect 87% of chicks (2 of 15 died), and the G8 construct performed even worse, protecting only 53% of the chicks (7 of 15 died). On the contrary, both the HVP413 and the G7 constructs were able to protect 100% of the chicks against the results of a severe NDV challenge infection.

### ILTV Challenge Infection

Protection against ILTV challenge infection was tested at 4 weeks p.v., and showed excellent protection by all the 4 rHVT-VP2-F-gD-gI vector vaccines tested: all 4 induced 100% protection. On the contrary, in the negative control group all 10 chicks displayed severe clinical signs of ILTV infection at day 4 or 5 post challenge: 7 of 10 died, and of the chicks left, 2 had to be euthanised.

Some further differentiation could be made in regard to the vaccine efficacy induced by the rHVT-VP2-F-gD-gI constructs, based on the limited clinical signs observed in these vaccinated groups: while the groups receiving the HVP412 or the HVP413 vaccine still had clinical scores of 20 respectively 37. The G7 vaccinated group only had a clinical score of 6.

Consequently, of the rHVT-VP2-F-gD-gI constructs tested for ILTV vaccination efficacy, the G7 construct (the rHVT according to the invention) performed the best, preventing almost all clinical signs of a severe ILTV challenge infection. This is a protection against ILTV challenge that is almost as good as that induced by the commercial vector vaccine HVT-ND-ILT, which in a similar trial typically leaves only a clinical score of 1.

### 4. Conclusions

The vaccination-challenge experiments described in Example 3 serve to confirm and expand upon the in vitro testing results of Example 1, and the in vivo testing and serology data described in Example 2. Combined these Examples provide for a complete characterisation of the different rHVT-VP2-F-gD-gI vector vaccine constructs tested, namely to demonstrate not only their effective replication and heterologous gene expression in vitro, and in vivo, but also their ability to induce an effective immune response that effectively protected target animals against infection and/or disease caused by a severe challenge infection by the respctive pathogens. Also, by testing them side by side in the same trial, their properties and respective vaccine efficacies could be carefully compared while under the same conditions.

Of the many rHVT-VP2-F-gD-gI constructs created and tested over time, only 4 were found to be genetically stable in vivo: HVP412, HVP413, G7, and G8. All of these provided 100% vaccination efficacy against a challenge infection with IBDV at 3 weeks p.v., and with ILTV at 5 weeks p.v. However against a challenge infection with a virulent NDV, only constructs HVP413 and G7 provided 100% protection at 5 weeks p.v.; the other two giving (much) less than 90% protection, which makes them ineffective NDV vaccines in practice. Among HVP413 and G7 further differentiation was possible based on the level of residual clinical signs observed after challenge: both after IBDV- and after ILTV challenge infection, the rHVT-VP2-F-gD-gI construct G7 left less residual clinical signs than did the HVP413 vector vaccine, i.e. the G7 construct protected more effectively than did the HVP413 construct.

In conclusion: of the many rHVT-VP2-F-gD-gI constructs made and tested, the construct G7, which is the rHVT according to the invention, had the most advantageous properties when considered overall. In most situations G7 even equalled the characteristics and vaccine properties of commercial rHVT vector vaccines carrying only two inserted heterologous genes. This is counter-intuitive and is better than could reasonably have been expected of a vector virus carrying 4 inserted heterologous genes.

### Legend to the figures

FIG. 1: Graphic representation of exemplary rHVT vector construct according to the invention:

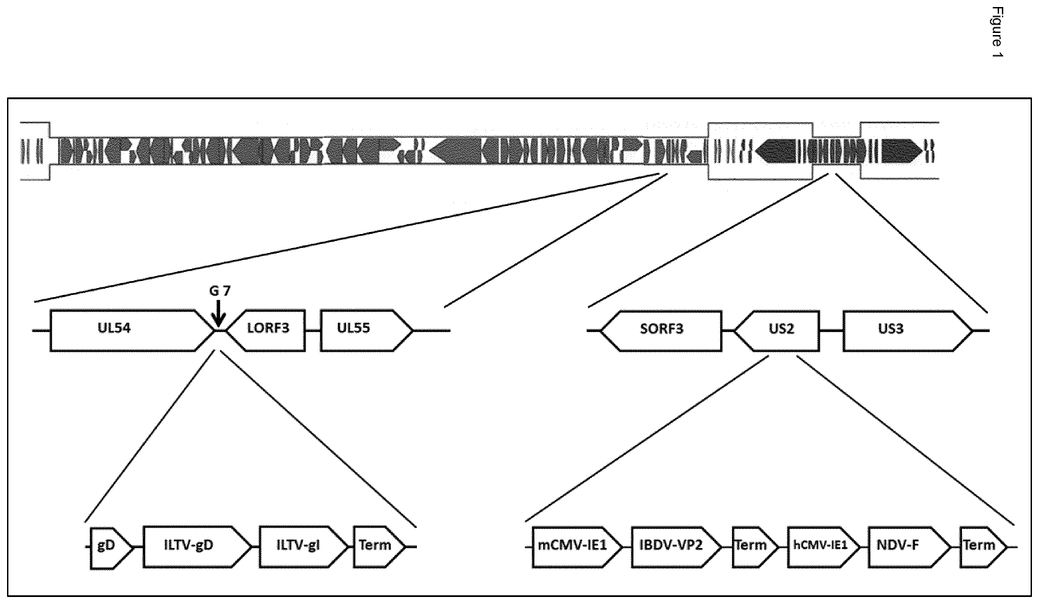

Along the top is indicated the HVT genome with its many ORFs indicated by block-arrows. The thin-lined boxes indicate the repeat regions.

In the middle the regions of the HVT genome are enlarged where inserts were introduced: between UL54 and LORF3, and: in Us2.

At the bottom are displayed examples of the two expression cassettes that were inserted;
bottom left: gD gene promoter-gD gene (comprising gI gene promoter)-gI gene (comprising gD gene terminator)-FHV1 Us9 gene terminator.
bottom right: mCMV-IE1 gene promoter-IBDV VP2 gene-SV40 late gene terminator-hCMV IE1 gene promoter-NDV F gene-hCMV-IE1 gene terminator.

1. A recombinant herpesvirus of turkeys (rHVT) comprising and expressing a first expression cassette which is inserted in the unique short (Us) region of the genome of the rHVT, and a second expression cassette which is inserted in the unique long (UL) region of the genome of the rHVT located between the UL54 and the LORF3 genes, the first expression cassette comprises an infectious bursal disease virus (IBDV) viral protein 2 (VP2) gene and a Newcastle disease virus (NDV) fusion (F) protein gene, and and the second expression cassette comprises a glycoprotein D and a glycoprotein I (gD and gI) gene of infectious laryngotracheitis virus (ILTV).

2. The rHVT according to claim 1, wherein the first expression cassette comprises in 5′ to 3′ direction and in this order:
a. a murine cytomegalovirus immediate early 1 (mCMV-IE1) gene promoter,
b. an IBDV VP2 gene,
c. a first transcription terminator,
d. a human cytomegalovirus immediate early 1 (hCMV-IE1) gene promoter,
e. an NDV F protein gene, and
f. a second transcription terminator,
wherein the mCMV-IE1 promoter and the first transcription terminator are operatively linked to the VP2 gene, and the hCMV-IE1 promoter and the second transcription terminator are operatively linked to the F gene.

3. The rHVT according to claims 1, wherein the first expression cassette is inserted in the Us2 gene in the Us region of the genome of the rHVT.

4. The rHVT according to claim 1, wherein the second expression cassette comprises in 5′ to 3′ direction and in this order:
a. an ILTV gD gene with an upstream promoter and a downstream terminator, and
b. an ILTV gI gene with an upstream promoter and a downstream terminator,
and whereby the promoters and terminators are operatively linked to the gD gene respectively to the gI gene.

5. A method for the construction of the rHVT of claim 1 comprising the insertion of the first and the second expression cassettes into a region of the genome of an HVT as defined in claim 1.

6. A host cell comprising the rHVT according to claim 1.

7. An immunogenic composition comprising the rHVT of claim 1, or the host cell according to claim 6, or both the rHVT of claim 1 and the host cell of claim 6.

8. A vaccine for poultry comprising the rHVT of claim 1, or the host cell according to claim 6, or both the rHVT of claim 1 and the host cell of claim 6, and a pharmaceutically acceptable carrier.

9. The vaccine according to claim 8, comprising at least one additional immunoactive component.

10. A method for producing a vaccine for poultry comprising the steps of:
a. infecting host cells in vitro with the rHVT of claim 1,
b. culturing and harvesting the infected host cells, and
c. admixing the harvested infected host cells with a pharmaceutically acceptable carrier.

11. (canceled)

12. A method for inducing an immune response in poultry against a virus selected from the group consisting of MDV, IBDV, NDV, and ILTV, comprising administering to a poultry the vaccine of claim 8.

13. The method of claim 12, wherein the method protects against, or reduces infection by one or more of MDV, IBDV, NDV, and ILTV in the poultry.

14. A method of vaccination of poultry to prevent or reduce infection by one or more of MDV, IBDV, NDV, and ILTV, the method comprising the step of inoculating said poultry with the vaccine according to claim 8.

15. The vaccine of claim 8, produced by the method of claim 10.
